(19) United States
(12) Patent Application Publication
(10) Pub. No.: US 2019/0212340 A1
(43) Pub. Date: Jul. 11, 2019

(54) AN ELISA-LIKE ASSAY FOR QUANTIFYING ENZYMATIC ACTIVITIES OF MONO-ADP-RIBOSYLTRANSFERASES

(71) Applicant: University of Southern California, Los Angeles, CA (US)

(72) Inventors: Yong Zhang, Los Angeles, CA (US); Jingwen Chen, Los Angeles, CA (US)

(21) Appl. No.: 16/336,074

(22) PCT Filed: Sep. 27, 2017
(86) PCT No.: PCT/US2017/053844

§ 371 Date: Mar. 22, 2019

Related U.S. Application Data
(60) Provisional application No. 62/400,566, filed on Sep. 27, 2016.

(51) Int. Cl. G01N 33/573

(52) U.S. Cl. CPC G01N 33/573

(57) ABSTRACT

Provided herein is a new molecular tool and assay that is useful to quantitatively determine the enzymatic activities of mono-ADP-ribosyltransferases, which are important regulators in various cellular events and implicated in many human diseases, therefore are promising drug targets. The methods of this disclosure enable sensitive and accurate quantify the activities of this group of enzymes and permits establishment of high-throughput screening assays for identification of inhibitors and activators of those enzymes for drug discovery and development.

### CROSS-REFERENCE TO RELATED APPLICATIONS

This application claims the benefit under 35 U.S.C. § 119(e) of U.S. Provisional Ser. No. 62/400,566, filed Sep. 27, 2016, the contents of which are hereby incorporated by reference into the present disclosure.

### BACKGROUND

Mono ADP-ribosylation is a reversible post-translational modification event mediated by mono ADP-ribosyltransferases. Single unit of ADP-ribose is conjugated to target proteins involved in major cellular processes such as DNA replication, cell proliferation, cell signaling and apoptosis. Mono ADP-ribosyltransferases are able to use nicotinamide adenine dinucleotide (NAD⁺) as co-substrates to transfer ADP-ribose. Previous research has demonstrated that several enzymes with a conserved poly (ADP-ribose) polymerase (PARP) domain are able to function as mono ADP-ribosyltransferases, exemplified by PARP14, 15 and 16. They can also transfer single unit of ADP-ribose onto themselves in a process called automodification.

Macrodomains are highly conserved domain structures throughout the evolution spectrum. They bind robustly to NAD⁺ metabolites such as mono ADP-ribose and poly ADP-ribose on substrate proteins, forming potential interactions through conjugation. Both PARP14 and 15 are macrodomain containing proteins thus are also termed Macro-PARPs. In particular, PARP14 Macrodomain 2 has been found to be able to specifically recognize mono ADP-ribose both in vitro and in cells. PARP-15 macrodomains are less studied but a previous study has linked them to decrease in TK-driven transcription.

Dysregulation of mono ADP-ribosyltransferases has been associated with cancer progression and poor survival. Overexpression of PARP14 and 15 have been found in diffuse large B-cell lymphoma whereas PARP16 plays a role in regulating key stress response pathways. To date, the catalytic domain crystal structures of PARP14, 15 and 16 have been resolved but still little is known about the characteristics of these enzymes. Several pan PARP inhibitors have been in the market with more in clinical trials, but there are no specific inhibitors for the three enzyme studied here. Developing specific inhibitors therefore has become important as it allows us to further understand cellular functions of each enzyme. A major difficulty faced during the inhibitor development phase is the highly conserved PARP catalytic domain where most current inhibitors can bind to.

A variety of methods have been developed for measuring the degree of poly ADP-ribosylation both in vitro and in cultured cells. However methods for detecting mono ADP-ribosylation are limited. A recent study has developed a fluorescence-based method to monitor mono ADP-ribosyltransferases activities through measuring the decrease in fluorescence signal. Measurement of such decrease may be not be sensitive to changes in enzymatic activity thus limits only to function to screen for inhibitors. Therefore, establishing a more sensitive and uniformed assay for multiple purposes is essential. This disclosure satisfies this need and provides related advantages as well.

### SUMMARY

ADP-ribosyltransferases (ARTs) catalyze reversible additions of mono- and poly-ADP-ribose onto diverse types of proteins by using nicotinamide adenine dinucleotide (NAD⁺) as a cosubstrate. In human ART superfamily, 14 out of 20 members are shown to catalyze endogenous protein mono-ADP-ribosylation and play important roles in regulating various physiological and pathophysiological processes. Identification of new modulators of mono-ARTs can thus potentially lead to discovery of novel therapeutics. This disclosure reports the development of a macrodomain-linked immunosorbent assay (MLISA) for characterizing mono-ARTs. By way of example, recombinant macrodomain 2 from poly-ADP-ribose polymerase 14 (PARP14) was generated with a C-terminal human influenza hemagglutinin (HA) tag for detecting mono-ADP-ribosylated proteins. Coupled with an anti-HA secondary antibody, the generated HA-tagged macrodomain 2 revealed high specificity for mono-ADP-ribosylation catalyzed by distinct mono-ARTs. Kinetic parameters of PARP 15-catalyzed automodification were determined by MLISA and in good agreement with previous studies.

Thus, in one aspect, provided herein is a method for detecting and/or quantifying mono- or poly-adenosine diphosphate ribose (ADP) transferase activity in an in vitro sample by measuring a detectable signal, the method comprising: (1) contacting a) a recombinant macrodomain peptide comprising an epitope for antibody recognition, b) an effective amount of a protein labeled with an ADP-ribose unit by ADP-ribosyltransferase, c) an effective amount of adenosine diphosphate ribose (ADP)-ribosyltransferase, d) an effective amount of nicotinamide adenine dinucleotide (NAD⁺), and e) an effective amount of a detectably labeled antibody that binds the recombinant macrodomain peptide with the epitope for antibody recognition, the contacting being under conditions that favor the formation of a complex comprising the detectably labeled antibody epitope bound to the recombinant macrodomain peptide that binds to a protein labeled with the ADP-ribose unit by ADP-ribosyltransferase. Subsequently the complex is contacted with an effective amount of a detectably labeled signal reagent; and the detectable signal derived from the detectably labeled signal reagent is measured, thereby detecting and/or quantifying mono- or poly-adenosine diphosphate ribose (ADP) transferase activity in the in vitro sample.

### BRIEF DESCRIPTION OF THE DRAWINGS

FIG. 1A is a general scheme of an ELISA-like assay for quantitatively determining enzymatic activity of mono-ADP-ribosyltransferase (ART).

FIG. 1B depicts a general scheme of MLISA. Five washes of PBST were conducted in between each step. 3% BSA, macrodomain 2 (M2), and anti-HA antibody-HRP conjugate were diluted in PBS, pH 7.4.

FIG. 1C shows SDS-PAGE gel of purified macrodomain 2 and PARP enzymes stained with Coomassie blue. Proteins loaded were normalized to 2 pig per well. Lanes 1-4: protein ladder, macrodomain 2, PARP14, and PARP 15.

FIG. 2 graphically shows the specificity of individual components in ELISA.

FIGS. 3A-3C show fluorescence intensity at various enzyme concentrations and reaction time points. 3A) PARP14; 3B) PARP15; 3C) PARP16.

FIG. 4 graphically shows enzyme kinetic parameters of PARP15-catalyzed automodification. Reaction rates (y-axis) measured by MLISA were plotted against varied NAD⁺ concentrations (x-axis) used in the reactions. The values were calculated as mean values of triplicates±SD.

FIG. 5 shows the DMSO effect on PARP14 and 15 enzymatic activities.

FIGS. 6A-6B shows the results of an inhibitor screening. 6A) PARP14; 6B) PARP 15.

FIGS. 7A-7B show that Macrodomain 2 (M2) is specific for mono-ADP-ribosylation by (7A) PARP15 and (7B) PARP14. Lane 1: anti-HA antibody-HRP conjugate (Ab) has no binding to BSA. Lane 2: M2 has no binding to BSA as examined by Ab. Lane 3: M2 has low to moderate binding to non-ADP-ribosylated PARPs as examined by Ab. Lane 4: Ab has low binding to ADP-ribosylated PARPs in the absence of M2. Lane 5: M2 has no binding to NAD⁺ in the assay system. Lane 6: M2 specifically binds to ADP-ribosylated PARPs from 2-hour reactions as measured by Ab. Values displayed were calculated as mean values of triplicates ±SD.

FIG. 8 graphically shows percentages of the background fluorescence intensities (control reactions without NAD⁺) relative to those of 2-hour PARP 14-catalyzed ([E]=3, 6, and 9 μM) automodifications.

FIGS. 9A-9C show frequency distribution of the maximal (2-hour reaction) and minimal (no reaction) fluorescence intensities for (9A) PARP15 ([E]=500 nM), (9B) PARP14 ([E]=3 μM), and (9C) PARP14 ([E]=6 μM). The data were from five different plates performed on two different days.

FIGS. 10A-10F show time- and concentration-dependent PARP-catalyzed mono-ADP-ribosylations as measured by MLISA. (10 A-C) PARP15: 500 nM, 1 μM, and 2 μM, M2: 0.1 μM; (10 D-F) PARP14: 3 μM, 6 μM, and 9 μM, M2: 0.3 μM. Fluorescence intensities at various enzyme concentrations were measured at 5-, 10-, 20-, 30-40-, 50-, and 60-minute time points for PARP15 and at 0.5-, 1-, 2-, 3-, 4-, 5-, and 6-hour time points for PARP14. The values were calculated as mean values of triplicates ±SD.

FIGS. 11A-11H show inhibition of PARP15 by individual compounds at varied concentrations. Normalized values were expressed as mean values of triplicates ±SD. (FI′: fluorescence intensity in the presence of inhibitors; FI₀: fluorescence intensity in the absence of inhibitors).

FIGS. 12A-12H show inhibition of PARP14 by individual compounds at varied concentrations. Normalized values were expressed as mean values of triplicates ±SD. (FI′: fluorescence intensity in the presence of inhibitors; FI₀: fluorescence intensity in the absence of inhibitors).

### DETAILED DESCRIPTION

### Definitions

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “purified” refers to a composition being substantially free from contaminants. With respect to polynucleotides and polypeptides, purified intends the composition being substantially free from contamination from polynucleotides or polypeptides with different sequences. In certain embodiments, it also refers to polynucleotides and polypeptides substantially free from cell debris or cell culture media.

The term “recombinant” refers to a form of artificial DNA that is created by combining two or more sequences that would not normally occur in their natural environment. A recombinant protein is a protein that is derived from recombinant DNA.

The term “binding” or “binds” as used herein are meant to include interactions between molecules that may be covalent or non-covalent which, in one embodiment, can be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

It is to be inferred without explicit recitation and unless otherwise intended, that when the present invention relates to a polypeptide, protein, polynucleotide or antibody, an equivalent or a biologically equivalent of such is intended within the scope of this invention.

As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “non-contiguous” refers to the presence of an intervening peptide, nucleotide, polypeptide or polynucleotide between a specified region and/or sequence. For example, two polypeptide sequences are non-contiguous because the two sequences are separated by a polypeptide sequences that is not homologous to either of the two sequences. Non-limiting intervening sequences are comprised of at least a single amino acid or nucleotide.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

Various proteins are also disclosed herein with their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

“Mono-ADP ribosyltransferases activity refers to the commonly catalyzation of the addition of ADP-ribose to arginine side chains using a highly conserved R-S-EXE motif. The reaction proceeds by breaking the bond between nicotinamide and ribose to form an oxonium ion. Through a series of reactions, nicotinamide is released. The modification can be reversed by ADP-ribosylhydrolases, which cleave the N-glycosidic bond between arginine and ribose to release ADP-ribose and unmodified protein: NAD+ is not restored by the reverse reaction.

“Poly adenosine diphosphate ribose (ADP) transferase activity” intends the activity of Poly-(ADP-ribose) polymerases (PARPs) that are found mostly in eukaryotes and catalyze the transfer of multiple ADP-ribose molecules to target proteins. As with mono-ADP ribosylation, the source of ADP-ribose is NAD⁺. PARPs use a catalytic triad of His-Tyr-Glu to facilitate binding of NAD⁺ and positioning of the end of the existing poly-ADP ribose chain on the target protein; the Glu facilitates catalysis and formation of a (1->2) O-glycosidic linkage between two ribose molecules. There are several other enzymes that recognize poly-ADP ribose chains, hydrolyse them or form branches.

As used herein, the term “macrodomain peptide” intends a module of about 180 amino acids which can bind ADP-ribose, an NAD metabolite, or related ligands. Binding to ADP-ribose can be either covalent or non-covalent in certain cases it is believed to bind non-covalently, while in other cases it appears to bind both non-covalently through a zinc finger motif, and covalently through a separate region of the protein. Macro domain proteins can be found in eukaryotes, and in bacteria, in archaea and in ssRNA viruses. These domains are known in the art and several examples of which are provided herein.

As used herein, the term “ADP-ribose unit” intends a compound comprising, consisting essentially of, or yet further consisting of a adenosine and a ribose joined through a diphosphate group.

“Adenosine diphosphate ribose (ADP) ribosyltransferase activity” intends the intracellular action of the addition of one or more ADP-ribose moieties to a protein. It is a reversible post-translational modification that is involved in many cellular processes, including cell signaling, DNA repair, gene regulation and apoptosis. Improper ADP-ribosylation has been implicated in some forms of cancer.

“Nicotinamide adenine dinucleotide (NAD) (also known as diphosphopyridine nucleotide (DPN+) and Coenzyme I) intends the coenzyme found in all cells. The compound is a dinucleotide, and it consists of two nucleotides joined through their phosphate groups. groups. The chemical structure is provide below.

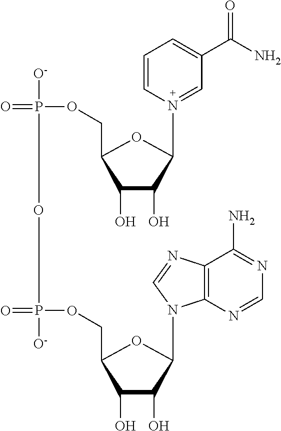

A “signal reagent” intends an agent (chemical, biological or otherwise) that emits a detectable signal.

The term “ADP-ribosyltransferase inhibitor” intends a molecule or an agent that inhibits the activity of ADP-ribosyltransferease.

The term “ADP-ribosyltransferase inhibitor” intends a molecule and agent that inhibits the activity of ADP-ribosyltransferease.

Poly (ADP-ribose) polymerase (PARP) is a family of proteins involved in a number of cellular processes such as DNA repair, genomic stability, and programmed cell death. The PARP family comprises 17 members. PARP is composed of four domains of interest: a DNA-binding domain, a caspase-cleaved domain, an auto-modification domain, and a catalytic domain. The DNA-binding domain is composed of two zinc finder motifs. In the presence of damaged DNA (base pair-excised), the DNA-binding domain will bind the DNA and induce a conformational shift. It has been hypothesized that this binding occurs independent of the other domains. The auto-modification domain is responsible for releasing the protein from the DNA after catalysis.

Macrodomains are conserved peptides of about 25 kDa with globular domains which harbor the ability to interact with the NAD⁺-derived, the post-translational modifications (PTMs) mono(ADP-ribose) and poly(ADP-ribose) (PAR). (Chen et al., Nat. Struct. Mol. Bio. 2014, 21(11):981-989). These PTMs are catalyzed by a family of ADP-ribosyltransferases, such as PARP-1, PARP-9, PARP-14 and PARP-15. The macrodomains are known in the art and examples of such are provided herein. PARP-1 has established roles in both DNA damage responses and transcriptional regulation. Several macrodomain-containing proteins are recruited to sites of PARP-1 activity. Additionally, the functions of macrodomain-containing proteins are often linked to their ability to interact with PARP-1-catalyzed PAR chains As reported by Chen et al. (2014), supra, macroH2A1.1 and macroH2A1.2 are splice variants of the same gene; each bearing a unique exon. Chen et al. (2014) reports that like most macrodomains, macroH2A1.1's macrodomain can interact with NAD-derived ligands, such as PAR, while the macrodomains of macroH2A1.2 and macroH2A2 cannot. Representative sequences of macroH2A1.1 are disclosed at GenBank Accession Nos. NP_613258.2, NP_613075.1, NP_001035248.1 (each last accessed on Sep. 26, 2017). Antibodies that specifically recognize and bind the peptide can be prepared using known methods or are commercially available from Abcam (ab37264) and Cell Signaling Technology (#4160). Representative macroH2A1.2 peptide sequences are disclosed at GenBank Accession Nos. BAB68541.1, NP_036145.1, and NP_001152985.1, (each last accessed on Sep. 26, 2017). Antibodies that specifically recognize and bind the peptide can be prepared using known methods or are commercially available from Abcam (#48275) and Millipore Sigma (#MABE61).

A representative sequence of PARP14 macrodomain1 can be found at GenBank Accession No. 3Q6Z_A (last accessed on Sep. 26, 2017) or another example is KCFSRTVLAPGVVLIVQQGDLARLPVDVVVNASNEDLKHYGGLAAALSKAAGPELQ ADCDQIVKREGRLLPGNATISKAGKLPYHHVIHAVGPRWSGYEAPRCVYLLRRAVQ LSLCLAEKYKYRSIAIPAISSGVFGFPLGRCVETIVSAIKENFQFKKDGHCLKEIYLVD VSEKTVEAFAEAVKTVF. A representative sequence of PARP14 macrodomain2 can be found at GenBank Accession No. 3Q71_A (last accessed on Sep. 26, 2017) or infra. A representative sequence of PARP14 macrodomain3 is found at GenBank Accession No. 5ABL_A and 4ABK_A (last accessed on Sep. 26, 2017) or is: DSGVYEMKIGSIIFQVASGDITKEEADVIVNSTSNSFNLKAGVSKAILECAGQNVERE CSQQAQQRKNDYIITGGGFLRCKNIIHVIGGNDVKSSVSSVLQECEKKNYSSICLPAIG TGNAKQHPDKVAEAIIDAIEDFVQKGSAQSVKKVKVVIFLPQVLDVFYANMKKRE.

Antibodies that specifically recognize and bind the peptide can be prepared using known methods or are commercially available from various sources such as Abcam, GeneTex, and Santa Cruz Biotechnology.

A representative sequence of PARP15 is provided herein and additional sequences are available at GenBank Accession No. 165631 and at uniProtKB-Q460N3 (each last accessed on Sep. 26, 2017). Antibodies that specifically recognize and bind the peptide can be prepared using known methods or are commercially available from various sources such as Abcam, GeneTex, and Santa Cruz Biotechnology.

A representative sequence of PARP9 is provided herein and additional sequences are available at GenBank Accession No. 165631 and at uniProtKB-Q460N3 (each last accessed on Sep. 26, 2017). Antibodies that specifically recognize and bind the peptide can be prepared using known methods or are commercially available from various sources such as Abcam, GeneTex, and Santa Cruz Biotechnology.

A representative sequence of PARP16 is provided herein and additional sequences are available at GenBank Accession Nos. NP_001303872.1 and NP001303873.1, as well as uniProtKB-Q8M5Y8-1, -2 and -3 (each last accessed on Sep. 26, 2017). Antibodies that specifically recognize and bind the peptide can be prepared using known methods or are commercially available from various sources such as Abcam, GeneTex, and Santa Cruz Biotechnology.

A representative sequence of ALC1 that binds to ADP-ribose is: SAELDYQDPDATSLKYVSGDVTHPQAGAEDALIVHCVDDSGHWGRGGLFTALEKRS AEPRKIYELAGKMKDLSLGGVLLFPVDDKESRNKGQDLLALIVAQHRDRSNVLSGIK MAALEEGLKKIFLAAKKKKASVHLPRIGHATKGFNWYGTERLIRKHLAARGIPTYIY YFPRSKSAVLHAQSSSSSSRQLVP. Antibodies that specifically recognize and bind the peptide can be prepared using known methods.

A representative sequence of MacroD is available at GenBank Accession No. NM_014067 (last accessed on Sep. 26, 2017). Antibodies that specifically recognize and bind the peptide can be prepared using known methods or are commercially available from various sources such as Abcam, Sigma-Aldrich and Novus Biologicals.

A representative sequence of MacroD2 is available at GenBank Accession Nos. NM_001033087 and NM_080676 (last accessed on Sep. 26, 2017). Antibodies that specifically recognize and bind the peptide can be prepared using known methods or are commercially available from various sources such as Sigma-Aldrich, Antibody Resource and Abmart.

A representative sequence of GDAP2 is available at UniProt Q9NXN4-1 and Q9NXN4-22 (last accessed on Sep. 26, 2017). Antibodies that specifically recognize and bind the peptide can be prepared using known methods or are commercially available from various sources such as Invitrogen, Abcam, and Novus Biologicals.

A representative sequence of C6orf1130 that binds ADP-ribose is: ASSLNEDPEGSRITYVKGDLFACPKTDSLAHCISEDCRMGAGIAVLFKKKFGGVQEL LNQQKKSGEVAVLKRDGRYIYYLITKKRASHKPTYENLQKSLEAMKSHCLKNGVTD LSMPRIGCGLDRLQWENVSAMIEEVFEATDIKITVYTL. Antibodies that specifically recognize and bind the peptide can be prepared using known methods.

As used herein, the term “detectable label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue®, and Texas Red®. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “contacting” intends bringing the reagents into close proximity with each other so that a chemical or biochemical reaction can occur among the reagents. In one aspect, the term intends admixing the components, either in a reaction vessel or on a plate or dish.

An “effective amount” intends an amount or quantity necessary to achieve a desired result. As is apparent to the skilled artisan, the effective amount will vary with the quality and identity of the reagents and the purpose of the assay or method.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

A “biologically active agent” or an active agent of this invention intends one or more of an isolated or recombinant polypeptide, an isolated or recombinant polynucleotide, a vector, an isolated host cell, or an antibody, as well as compositions comprising one or more of same.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous supply of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin. a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

### Modes for Carrying Out the Disclosure

This disclosure provides methods for detecting and/or quantifying mono- or poly-adenosine diphosphate ribose (ADP) transferase activity in an in vitro sample by measuring a detectable signal. The method comprises, or alternatively consists essentially of, or yet further consists of the following series of steps:

(1) contacting:
a) a recombinant macrodomain peptide comprising an epitope for antibody recognition,
b) an effective amount of a protein labeled with an ADP-ribose unit by ADP-ribosyltransferase,
c) an effective amount of adenosine diphosphate ribose (ADP)-ribosyltransferase,
d) an effective amount of nicotinamide adenine dinucleotide (NAD⁺), and
e) an effective amount of a detectably labeled antibody that binds the recombinant macrodomain peptide with the epitope for antibody recognition,
the contacting being under conditions that favor the formation of a complex comprising the detectably labeled antibody bound to the recombinant macrodomain peptide that binds to a protein labeled with the ADP-ribose unit by ADP-ribosyltransferase;

(2) contacting the complex with an effective amount of a detectably labeled signal reagent; and

(3) measuring the signal derived from the detectably labeled signal reagent, thereby detecting and/or quantifying mono- or poly-adenosine diphosphate ribose (ADP) transferase activity in the in vitro sample.

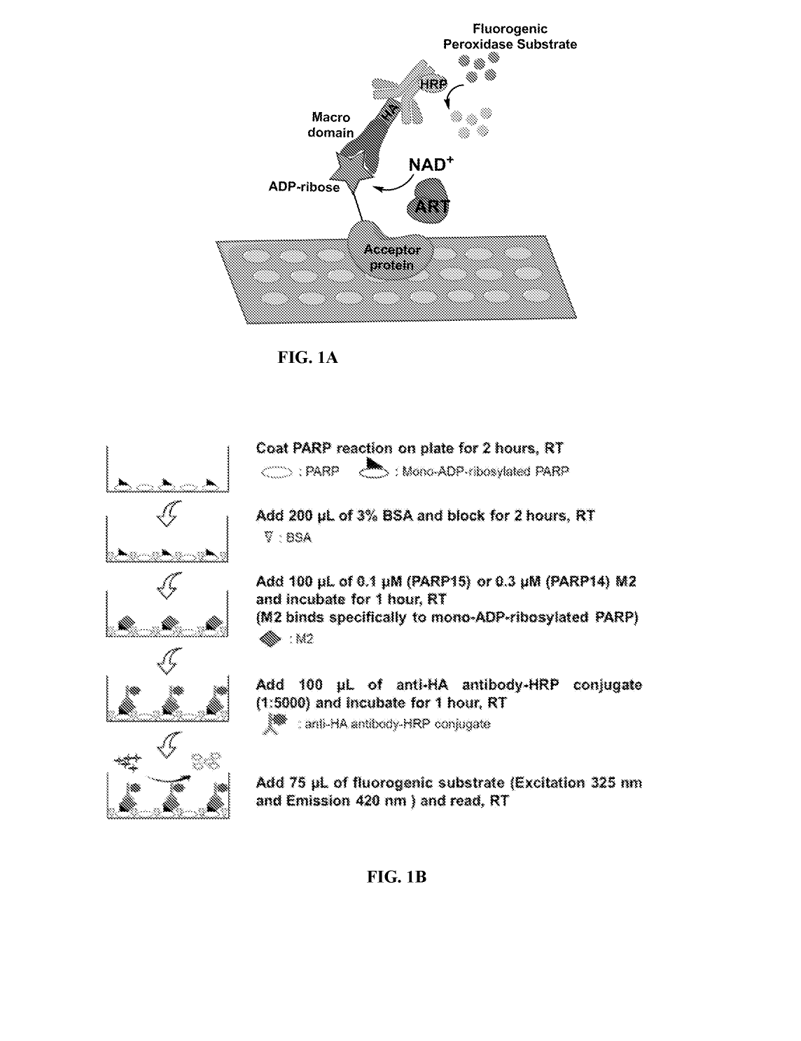

FIG. 1A graphically show the interaction of the reagents of the disclosed method. As disclosed herein, the Macro-domain is recombinantly joined to a C-terminal tag, e.g., HA tag. An exemplary polynucleotide encoding the Marcodomain peptide is provided herein. The polynucleotides encoding the Marcodomain containing peptides are recombinantly expressed in a host cell, such as an E. coli BL21 (DE3).

Also as depicted in FIG. 1A, the method of this disclosure can be conducted in a high-throughput manner using, for example, 96-well plates. These steps are graphically depicted in FIG. 1B. As depicted in FIG. 1B and described herein, the above method can be modified by the addition of an effective amount of a blocking agent and washes. The reactions can be conducted at room temperature and for an effective amount of time to effect specific binding.

Non-limited examples of the detectable signal of the detectably labeled signal reagent is from the group of: a colorimetric signal, a chemiluminescent signal, or a fluorescent signal, such as for example, QuantaBlu™ fluorogenic peroxidase substrate and fluorescence intensity can be measured using a Synergy H1 Multi-Mode Reader.

Non-limiting examples of epitopes for antibody recognition are selected from the group of: HA tag, FLAG tag, His6 tag, myc-tag or a peptide or any other agent that can be genetically fused with the macrodomain and recognized by their respective mono- and poly-clonal antibodies.

In another aspect, the ADP-ribosyltransferase enzyme is a PARP enzyme, non-limiting examples of such include a macrodomain derived from a human macro-domain containing protein selected from the group of: MacroH2A1.1, MacroH2A 1.2, MacroH2A2, PARP14, 15, 16, or 9, ALC1, MacroD1, Macro D2, GDAP2, or C6orf130. In one aspect, the macrodomain is derived from a PARP protein selected from the group of PARP14, PARP15, or PARP9. In another aspect, the macrodomain is the M2 macrodomain, which is part of PARP14. As provided infra, the sequences of PARP14, PARP15, and PARP16 are catalytic domains (i.e. domains with ADP-ribosyltransferase activities). They are portions of their corresponding complete proteins. Also within the scope of this disclosure are equivalents of the PARP proteins. In a further aspect, the macrodomain is selected from the group of: macrodomain1, macrodomain2, or macrodomain3 derived from PARP14. Non-limiting exemplary sequences for the production of these elements are disclosed in the Sequence Listing disclosed below. In addition to the exemplary sequences, also intended within the scope of this disclosure are sequences are equivalent sequences as defined herein. An equivalent includes a polypeptide, protein or polynucleotide having at least 70%, or alternatively at least 80%, or alternatively at least 85%, or alternatively at least 90%, or alternatively at least 95%, or alternatively at least 97%, primary sequence (polynucleotide or amino acid sequence) identity with the reference sequence.

The method can be modified to identify and select potential reaction modifiers. In those aspects, the method further comprises, alternatively consists essentially of, or yet further consists of, contacting a test agent with the recombinant macrodomain peptide with an epitope for antibody recognition, the ADP-ribosyltransferease and NAD⁺. In one aspect, the test agent is a potential ADP-ribosyltransferase inhibitor. Further provided is a therapeutic method comprising, or alternatively consisting essentially of, or yet further consists of administration of an effective amount the reaction modifier and/or a composition containing the reaction modifier, to a patient in need thereof.

In a further aspect, the method further comprises quenching the reaction prior to contacting the complex with the effective amount of the detectably labeled signal reagent.

The method also can be modified by repeating the method steps with varying amounts of ADP-ribosyltransferase enzyme, and then further optionally comparing the results. In addition, the signal emitted from the detectably labeled signal reagent can be measured one or more times. Alternatively, the signal emitted from the detectably labeled signal reagent is measured multiple times and Km values are calculated using non-linear regression.

Kits are further provided herein. The kits comprise, or alternatively consist essentially of, or yet further consist of reagents for performing the methods as disclosed herein and optionally instructions for carrying out the methods.

### Experimental Materials and Methods

### Experiment No. 1

An immuno-based method is described that in addition to identifying inhibitors specificy for mono ADP-ribosyltransferases the method, also characterize kinetic parameters (FIG. 1A). This method incorporates Macrodomain 2 (M2) from PARP and related protens, e.g., PARP14, as a binding module for mono ADP-ribose generated through PARP automodification. The measurement of mono ADP-ribosylation is indicated by the increase of fluorescence intensity based on the level of automodified enzymes. The fluorescence-based method is compatible with high-throughput screening applications for fast inhibitor screening and can also be used for enzyme kinetic characterization. It provides a more accurate and sensitive alternative for multipurpose studies of mono ADP-ribosyltransferases with consistency comparing to the current method available.

The specificity of individual components in each step of the fluorescence-based assay was assessed through a set of control experiments performed in triplicate. For detection of mono ADP-ribosylation, Macrodomain 2 derived from PARP14 with HA tag was used as it is able to specifically detect Mono ADP-ribose. Mono-PARPs such as PARP14, 15 and 16 are able to automodify themselves on residues within the automodification domains with presence of NAD⁺. Specificity of M2 to Mono ADP-ribose on automodified enzymes is examined first. Macrodomain 2 concentration was optimized to 0.1 μM for PARP14 and PARP15 due to fast onset enzymatic activity and 1 μM was tested for PARP16 based on the low activity previously reported by (Karlberg, T., et al, Structural basis for lack of ADP-ribosyltransferase activity in poly(ADP-ribose) polymerase-13/zinc finger antiviral protein. The Journal of Biological Chemistry 2015, 290 (12), 7336-44). The fluorescence intensity of wells only containing PARP enzymes in reaction buffer without NAD⁺ and two-hour control was measured on a plate reader.

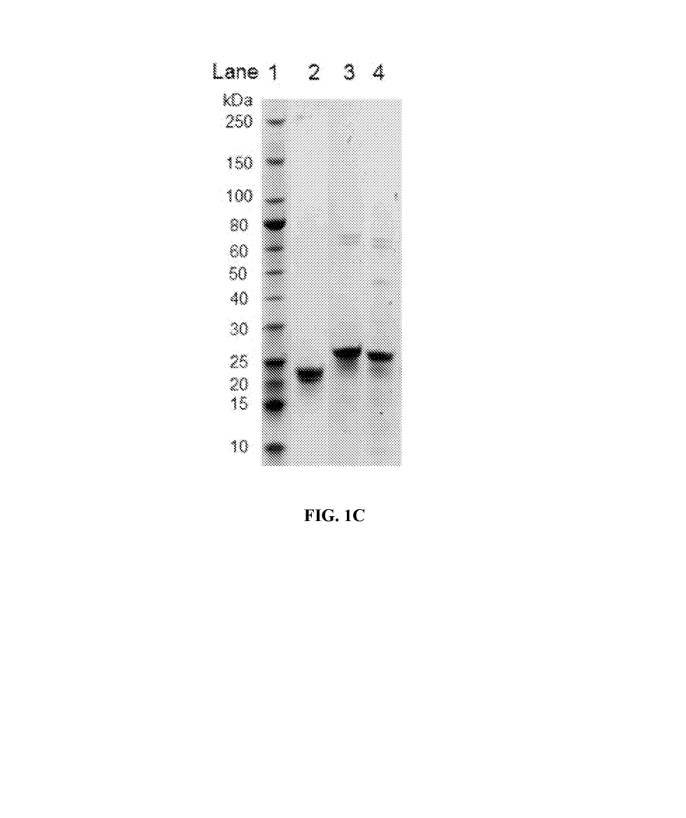

The results suggested that M2 is specific to automodified PARPs as the fluorescence intensity was significantly higher for two hour controls wells comparing to the No NAD+ wells (FIG. 2). The No NAD⁺ wells showed higher fluorescence intensity comparing to No M2 wells possibly due to domain-domain interactions in between the catalytic domain and M2 since they both derive from PARP14. 3% BSA has been determined to sufficiently block the gaps on the bottom of the wells and anti-HA HRP conjugate showed minimal binding to both BSA and PARP enzymes.

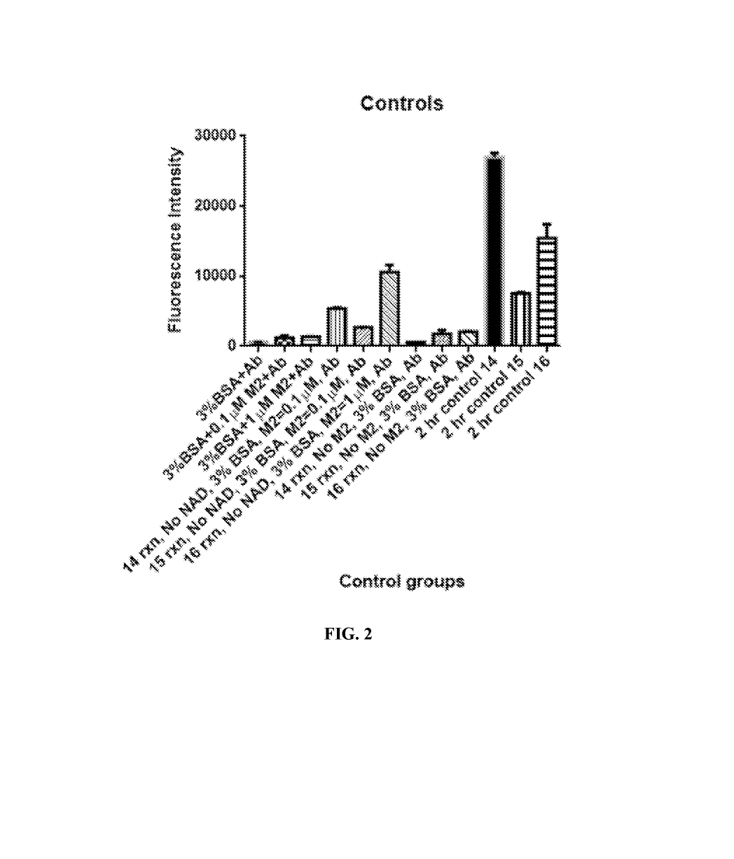

Next, the levels of automodification of PARP enzymes were tested to determine if they are dependent on enzyme concentration and length of reaction time. Three time points and three concentrations were tested for PARP14. All experimental wells at different concentrations and time points exhibited higher fluorescence intensity than that of No NAD⁺ control wells. The result demonstrated that wells with higher PARP14 concentrations showed higher fluorescence intensity, with the highest at an enzyme concentration of 3 μM and lowest at an enzyme concentration of 500 nM (FIG. 3A). This increase in fluorescence intensity was also time dependent, which is most obvious using an enzyme concentration of 3 μM, with fluorescence intensity highest at 6-hour time point versus lowest at 2-hour time point.

This experiment was repeated with PARP15. Conditions were mostly kept the same with minor variations such as lower enzyme concentration was used due to the fast activity of PARP15. Fluorescence intensity was highest with reaction time of 6 hours and an enzyme concentration of 2 μM (FIG. 3B). The increase in fluorescence intensity was much more significant than the increase observed in PARP14, suggesting that PARP15 may be more active in terms of enzymatic activity or have more automodification sites than PARP 14. PARP16 also was assayed. In order to retain consistency in experiments conducted with PARP14 and PARP15, a M2 concentration of 0.1 μM was used throughout the time and concentration study for PARP enzymes. For PARP16, much higher concentrations are required considering its relatively low activity reported (Karlberg, T. et al, Crystal structure of human ADP-ribose transferase ARTD15/PARP16 reveals a novel putative regulatory domain. The Journal of Biological Chemistry 2012, 287 (29), 24077-81). Fluorescence intensity in between control and experimental wells appeared to be similar possibly due to low activity resulted in less automodification comparing to PARP14 and 15 (FIG. 3C). The time dependent effect was most evident with 80 μM of enzyme concentration while enzyme concentration effect was more visible at 2 hour reaction time point. When using a high enzyme concentration of 160 μM, fluorescence intensity decreased as reaction time increased, suggesting potential precipitation of proteins due to temperature increase and loss of binding ability to the wells.

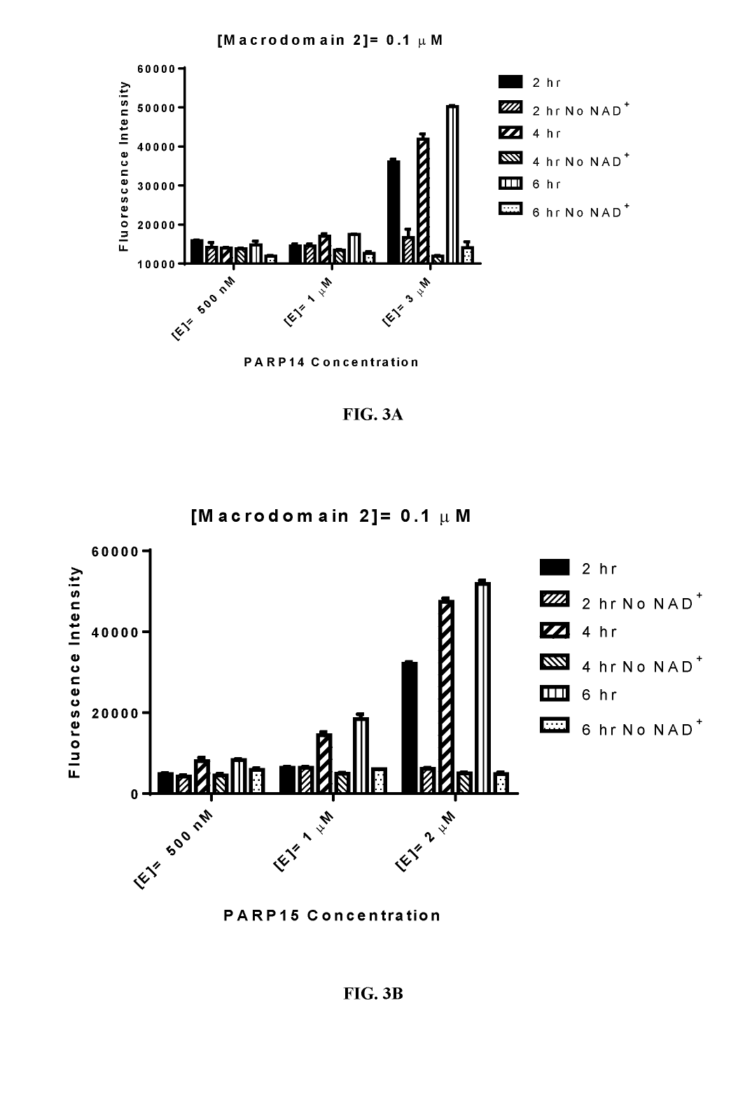

To evaluate the quality of M2-based ELISA in measuring kinetic characteristics of PARP enzymes, we chose PARP15 as the model enzyme since a published Km value was previously reported, (Karlberg, T., et al, Structural basis for lack of ADP-ribosyltransferase activity in poly(ADP-ribose) polymerase-13/zinc finger antiviral protein. The Journal of Biological Chemistry 2015, 290 (12), 7336-44). The quenching method using TCA was developed from a previous assay used for inhibitor screening (Venkannagari, H. et al Activity-based assay for human mono-ADP-ribosyltransferases ARTD7/PARP15 and ARTD10/PARP10 aimed at screening and profiling inhibitors. European journal of pharmaceutical sciences: (Official Journal of the European Federation for Pharmaceutical Sciences 2013, 49 (2), 148-56). PARP15 concentration was kept at 500 nM with NAD⁺ concentrations ranging from 5 μM to 4 mM. The reactions were quenched with 20% TCA at various time points and a standard curve was constructed by plating saturated automodified PARP15 along with the reaction wells on the same plate. The enzyme concentrations in standard curve wells were determined through Bradford assay and a linear correlation of automodified PARP15 enzyme concentration and fluorescence intensity therefore can be obtained. The automodified PARP15 concentrations in experimental wells were used for kinetics characterization and the calculated Km value of PARP15 is 7.148±3.138 μM comparing to the reported value of 5.8±1.9 μM (Karlberg, T., et al, Structural basis for lack of ADP-ribosyltransferase activity in poly(ADP-ribose) polymerase-13/zinc finger antiviral protein. The Journal of Biological Chemistry 2015, 290 (12), 7336-44.) (FIG. 4), this method is both sensitive and credible for kinetics study.

PARP inhibitors have now become therapeutic agents for disease treatment, especially in tumor progression. It is then necessary to evaluate the quality of this method in identifying potential hits for mono-PARP inhibitors. Selected PARP15 inhibitors at different concentrations were used to examine their inhibitory effect on PARP14 and 15 and to determine whether such inhibition is dose dependent.

TABLE 1
List of compounds used for inhibition studies.

| Number | Name | Structure |
|---|---|---|
| 1 | Olaparib | 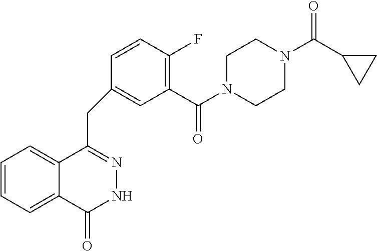 [SMILES: O=C(c1cc(Cc2n[nH]c(=O)c3ccccc23)ccc1F)N1CCN(C(=O)C2CC2)CC1] |
| 2 | XAV939 | 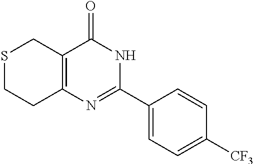 [SMILES: O=c1[nH]c(-c2ccc(C(F)(F)F)cc2)nc2c1CSCC2] |
| 3 | 1,5-Isoquinolinediol | 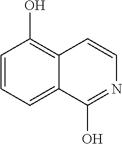 [SMILES: Oc1cccc2c(O)nccc12] |
| 4 | Minocin | 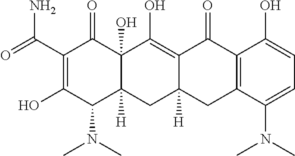 [SMILES: CN(C)c1ccc(O)c2c1C[C@H]1C[C@H]3[C@H](N(C)C)C(O)=C(C(N)=O)C(=O)[C@@]3(O)C(O)=C1C2=O] |
| 5 | DR2313 | 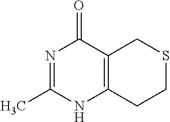 |
| 6 | 6(5H)- Phenanthridinone | 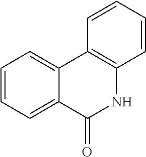 [SMILES: O=c1[nH]c2ccccc2c2ccccc12] |
| 7 | Nicotinamide | 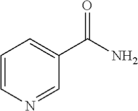 |
| 8 | Adenine | 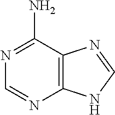 |

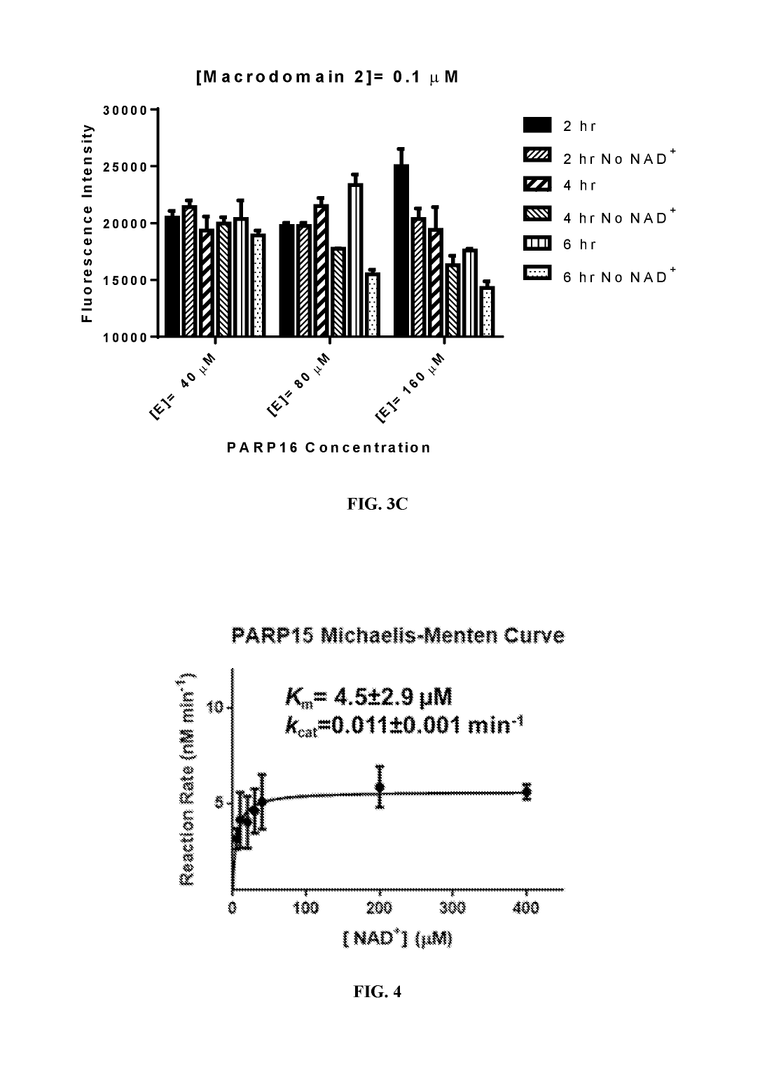

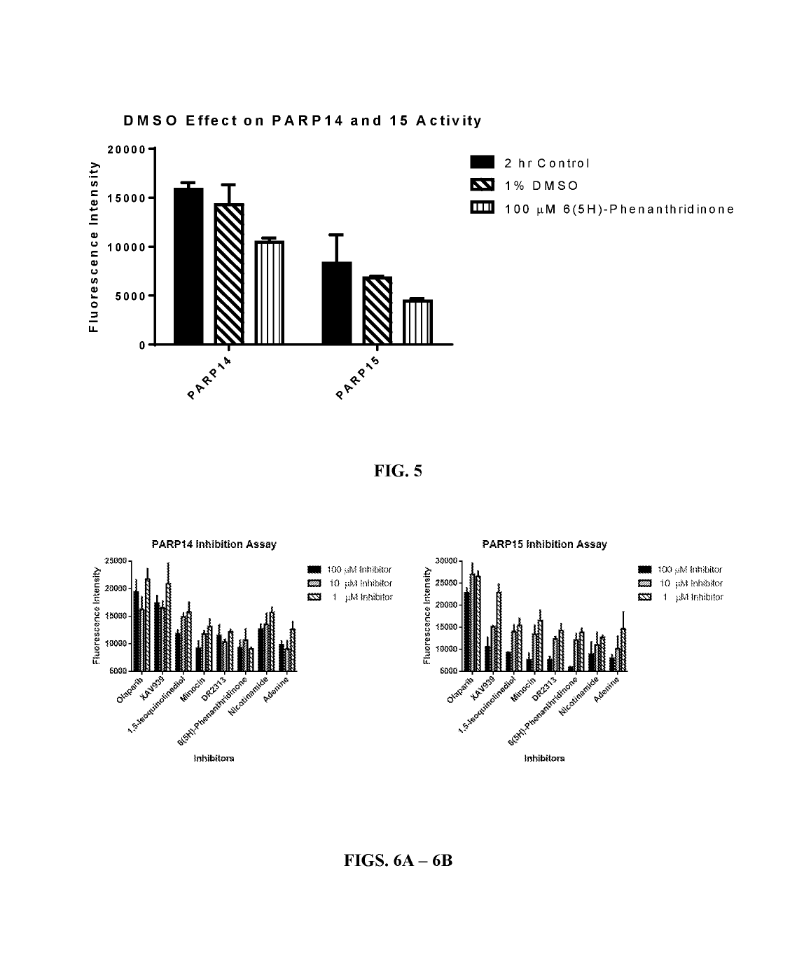

PARP14 was tested along with PARP15 for comparison based on the catalytic domain conservation. Depending on the water solubility of the inhibitors, they were dissolved in either DMSO or water and further diluted in water with a maximum 1% of DMSO in the solution. The interference of DMSO was evaluated comparing the fluorescence intensity of 1% DMSO wells to that of the 2-hr control wells and 100 μM inhibitor wells for both enzymes. DMSO slightly interfered with enzyme binding to the plate resulting in lower fluorescence intensity but it is not significant comparing to inhibition effect of 100 μM inhibitor (FIG. 5). As expected, PARP14 was inhibited and a dose dependent effect can be observed in some inhibitors but for others the optimal inhibitor concentration appeared to be 10 μM. PARP15 was also inhibited by all compounds tested and a dose dependent effect can be observed for all inhibitors except for Olaparib as the fluorescence intensity decreases and inhibitor concentration increases. Olaparib exhibited better inhibitory effect at 100 μM but it is less potent comparing to other inhibitor possibly due to the fact that it was developed as a PARP1 inhibitor, which has a similar but different catalytic domain from PARP15 (FIG. 6).

### Experimental Methods

### 1. Materials and Reagents

Wild-type human PARP14, PARP15 and PARP16 cDNA were obtained from GE Dharmacon (Lafayette, Colo.) (Accession number: BC039604, BC 101701 and BC031074). Exemplary sequences are provided herein. Synthetic DNA sequence of Macrodomain 2 (residue 983-1196) of PARP14 with codon optimized for bacterial expression was ordered from IDT (Coralville, Iowa).

Olaparib was purchased from Selleckchem (Houston, Tex.) and Minocin, XAV939, 1,5-Isoquinolinediol, DR2313, 6(5H)-Phenanthridinone, Nicotinamide, Adenine and β-NAD were purchased from Sigma Aldrich (St. Louis, Mo.). 96-well high-binding fluorescence plates (Greiner Bio-one), pureGrade microplates (BRANDTech Scientific) and Dithiothreitol (DTT) were purchased from VWR (Radnor, Pa.). Trichloroacetic acid (TCA) and Tris base were purchased from Fisher Scientific (Hampton, N.H.). Anti-HA monoclonal antibody-HRP conjugate, Coomassie Plus (Bradford) Assay Kit and QuantaBlu Fluorogenic Peroxidase Substrate were purchased from Thermo Fisher Scientific (Waltham, Mass.).

All chemicals used for coupled-enzyme assay and Glutamate Dehydrogenase from bovine liver were purchased from Sigma Aldrich. MBP-PncA (maltose binding protein fused to nicotinamidase from Salmonella enterica) expression construct was a generous gift from Dr. Jorge C. Escalante-Semerena (University of Georgia) and was purified through one-step Ni-NTA purification. Semi-micro Polystyrene Cuvettes were purchased through VWR (Radnor, Pa.).

### 2. PARP Enzymes and Macrodomain 2

### 1.1 Molecular Cloning

Primers (P1-2 and P8-11) for PARP14, PARP15 and PARP16 were first designed to amplify the catalytic domains with an N-terminal His6-tag and Factor Xa cleavage site (PARP14: residue 1611-1801; PARP15: 481-678; PARP16: 5-279). Primers (P3-P4) for Macrodomain 2 (residue 999-1196) were designed to amplify the Macrodomain 2 synthetic gene (Integrated DNA Technologies, Coralville, Iowa) with N-terminal His6-tag and Factor Xa cleavage site. Primers were then designed to incorporate XbaI and XhoI restriction enzyme sites at the 5′ and 3′ end (PARP14:P5-6;M2:P5,7;PARP15:P12-13;PARP16:P12,14). The catalytic domains of PARP enzymes were PCR-amplified as DNA inserts for double digestion. Macrodomain 2 was PCR-amplified with the same restriction enzyme sites and an N-terminal HA-tag. Constructs were generated through inserting amplified fragments into pET-28a+ vector with C-terminal His6-tag for expression obtained from Dr. Julio A Camarero's lab (University of Southern California). All expression constructs were verified by DNA sequencing (Genewiz, South Plainfield, N.J.)

### 1.2 Expression

Escherichia coli BL21 (DE3) cells were transformed with the generated constructs for protein expression in the E. coli in LB media with Kanamycin (50 μg/mL). The overnight bacterial culture grown at 250 rpm and 37 IC in an incubator shaker (Series 25, New Brunswick Scientific, N.J.) in 1 liter LB with Kanamycin (50 μg/mL) at 37° C. and induced with 0.5 mM Isopropyl 3-D-1-thiogalactopyranoside (IPTG) at a density (OD600 nm) of 0.6-0.8 at 22° C. overnight. Cells were harvested by centrifugation at 4,550 g (Beckman J6B Centrifuge, JS-4.2 rotor), resuspended in Equilibrium Buffer (20 mM Tris-HCl, pH 8.0, 200 mM NaCl and 20 mM imidazole) and lysed through a French Press (GlenMills, N.J.) at 25,000 psi for three times. Cell debris was removed by centrifugation at 27,000 g for 1 hour (Beckman Coulter centrifuge, JA-17 rotor) and supernatant was filtered through a 0.45 μm membrane. The filtrate was loaded on a gravity flow column packed with 1 ml Ni-NTA agarose resin (Thermo Fisher Scientific, Waltham, Mass.), followed by washing with 15 column volumes of Wash Buffer (20 mM Tris-HCl, pH 8.0, 200 mM NaCl and 30 mM imidazole). Proteins were then eluted in 15 column volumes of Elution Buffer (20 mM Tris-HCl, pH 8.0, 200 mM NaCl and 400 mM imidazole), dialyzed in Storage Buffer (20 mM Tris-HCl, pH 8.0, 300 mM NaCl, 1 mM dTT and 10%0/glycerol) at 4° C. for overnight and another 6 hours in fresh storage buffer, and concentrated through a Amicon centrifugal concentrator (Emd Millipore, Temecula, Calif.) with a 10 kDa cutoff. Purified proteins were aliquoted and flash-frozen in liquid nitrogen for storage at −80° C. Protein purity was assessed through Nanodrop (Thermo Fisher Scientific, Waltham, Mass.) and purity visualized through SDS-PAGE. Calculated Molecular extinction coefficient values for PARP 14, 15, 16, Macrodomain 2 and MBP-PncA are 1.196, 1.13, 1.14, 0.781 and 1.536.

### 3. ELISA Plate Assay

### 2.1 General Procedure

The general scheme of ELISA assay is illustrated in FIG. 1. PARP automodification reactions (100 μL) were conducted in 96-wells plate together with the coating process for 2 hours at room temperature. After five times of 200 μL PBST washes (0.1% v/v Tween 20 in PBS buffer, pH 7.4) per well the plates were blocked with 3% BSA dissolved in PBS buffer for 2 hours under room temperature followed by five washes of 200 μL PBST per well. The plates were incubated for 1 hour upon addition of 100 μL of 0.1 μM Macrodomain 2 and then washed five times with 200 μL PBST per well. 100 μL of 1:5000 anti-HA-HRP conjugate in PBS was added and plates were incubated for 1 hour at room temperature, followed by washing with 200 μL PBST per well for five times. The fluorescence signal was developed by addition of 75 μL QuantaBlu Fluorogenic Peroxidase Substrate and recorded by Synergy H1 Multi-Mode Reader (BioTek, VT). Kinetic parameters were calculated using nonlinear regression Michaelis-Menten model using GraphPad Prism (GraphPad Software). All experiments were performed at least in triplicate.

To control for any potential interactions within the reaction system, wells containing only PARP enzymes without NAD⁺ were analyzed together with experimental samples in triplicates on the same plate. Other control wells were also analyzed to assess any non-specific binding of the following system components: 1) Macrodomain 2 to PARP enzymes, 2) Macrodomain 2 to BSA and 3) HA-HRP conjugated antibodies to PARP enzymes and BSA.

Optimal enzyme concentration determined is as follows: PARP14 1 μM, PARP15 500 nM and PARP16 80 μM.

Optimal ELISA condition determined is as follows: 50 mM Tris-HCl, pH 7.4, 2 mM dTT, 500 μM of NAD⁺.

Above conditions were used to set up 2 hours control experiments. 2.2 Time and Concentration Dependent Assay Preparation

96-well ELISA plates were coated with reactions in triplicates consisting of various concentrations of PARP enzymes (PARP14: 500 nM, 1 μM and 3 μM; PARP15: 500 nM, 1 μM and 2 μM; PARP16: 40 μM, 80 μM and 160 μM) with 500 μM of NAD⁺ in Reaction Buffer (50 mM Tris-HCl, pH 7.4, 2 mM dTT) and incubated for 6, 4 and 2 hours under room temperature. The wells were washed five times with PBST (1×PBS buffer containing 0.1% v/v Tween 20).

### 2.3 Inhibition Assay Preparation

96-well ELISA plates were coated with reactions in triplicates consisting of PARP enzymes (PARP14: 1 μM; PARP15: 500 nM) with various inhibitors at different concentrations (1, 10 and 100 μM) in addition to 500 μM of NAD⁺ in Reaction Buffer (50 mM Tris-HCl, pH 7.4, 2 mM dTT). The reaction was incubated for 2 hours under room temperature followed by five washes of PBST. To control for any potential inhibition activity related to DMSO, wells containing 1% DMSO were analyzed in triplicates alongside with no inhibitor control wells and 100 μM control inhibitor wells.

### 2.4 Kₘ Determination Assay Preparation

### 2.4.1 Kₘ Determination

96-well ELISA plates were coated with reactions in triplicates consisting of 500 nM PARP15 with various concentrations of NAD⁺ (5, 10, 20, 30, 40, 200, 400, 1000, 2000 and 4000 μM) in Reaction Buffer (50 mM Tris-HCl, pH 7.4, 2 mM dTT). The reactions were quenched with 20% ice-cold TCA at various time points (0 to 20 minutes). The plates were further incubated for up to 2 hour under room temperature followed by five washes of PBST.

### 2.4.2 Standard Curve

PARP15 automodification assay was performed by adding 50 μg/ml PARP15 to Reaction Buffer with 500 μM NAD⁺ in a microcentrifuge tube for 2 hours. Automodified PARP15 was diluted and plated simultaneously on both the ELISA plate and clear pureGrade plate according to manufacturer's protocol. Upon addition of Coomassie Plus (Bradford) Assay Reagent the absorbance of each well was measured on a Synergy H1 Hybrid Multi-Mode Microplate Reader (BioTek, VT). The concentrations of automodified PARP15 in each well were determined through linear regression curve using BSA standards. A standard curve was constructed through a linear correlation of automodified PARP15 concentrations and the corresponding fluorescence signals.

This disclosure provides a macrodomain-linked immunosorbent assay (MLISA) as a generally applicable method for quantitative characterization of mono-ART enzymes. By way of example, exploiting macrodomain 2 of PARP14 that binds tightly to both free and protein-linked ADP-ribose in vitro and in cells while lacking hydrolase activity, (Rosenthal, F. et al. Nat. Struct. Mol. Biol. 20, 502-507, (2013); Forst, A. H. et al. Structure 21, 462-475, 19 (2013)). A recombinant agent for recognizing mono-ADP-ribosylated proteins was generated. In combination with an anti-hemagglutinin (HA) antibody, the macrodomain 2-based ADP-ribose binding module was shown to detect protein mono-ADP-ribosylation with good selectivity. As a general approach, the developed MLISA allows rapid quantification of protein ADP-ribosylations catalyzed by distinct mono-ARTs exemplified by PARP15 and PARP14, as well as characterization of PARP15 enzyme kinetics. Furthermore, a panel of commonly used chemical tools for PARPs was examined for inhibitory activities against PARP15 and PARP14 by performing MLISA-based screening in 96-well plates. As reported herein, MLISA provides a convenient and quantitative approach for characterizing mono-ARTs and potentially enables discovery of new mono-ARTs inhibitors in a high-throughput compatible format.

### Experimental No. 2

Experiment No. 2 is an expansion of the studies reported above.

### 2.1 Materials and Reagents.

cDNA of human PARP15 (accession number: BC101701) and PARP14 (accession number: BC039604), were obtained from GE Dharmacon (Lafayette, Colo.). Synthetic DNA encoding macrodomain 2 (residue 983-1196) of PARP14 with codon optimized for bacterial expression was purchased from Integrated DNA Technologies (IDT) (Coralville, Iowa).

Olaparib was purchased from Selleckchem (Houston, Tex.) and Minocin, XAV939, 1,5-isoquinolinediol, DR2313, 6(5H)-phenanthridinone, nicotinamide, adenine, O-NAD⁺, and β-NADH were purchased from Sigma Aldrich (St. Louis, Mo.). 96-well high-binding fluorescence plates were purchased from Greiner Bio-One (Monroe, N.C.). PureGrade microplates and semi-micro polystyrene cuvettes were purchases from BrandTech Scientific, Inc (Essex, Conn.). Dithiothreitol (DTT) was purchased from VWR International (Radnor, Pa.). Trichloroacetic acid (TCA) and Tris base were purchased from Fisher Scientific (Waltham, Mass.). Anti-HA monoclonal antibody-horseradish peroxidase (HRP) conjugate (clone 2-2.2.14), Pierce™ Coomassie Plus (Bradford) assay kit, and QuantaBlu™ fluorogenic peroxidase substrate were purchased from Thermo Fisher Scientific (Waltham, Mass.).

### 2.2 Molecular Cloning and Protein Expression and Purification.

The catalytic domains of PARP15 (residue 481-678) and PARP14 (residue 1611-1801), with N-terminal His₆-tags and Factor Xa cleavage sites were amplified through polymerase chain reaction (PCR) using primers P1-2 and P8-9 (Table 2), followed by additions of XhoI and XbaI restriction enzyme sites at 5′- and 3′-end, respectively, using primers listed in Table 2 (P5 and P6 for PARP14; P10 and P11 for PARP15).

TABLE 2
List of primer sequences used in molecular cloning.

| Name | Application | Sequence |
|---|---|---|
| P1 | PARP14 catalytic domain | CACCATCATCATCATCATATTGAGGGTCGC |
| | forward | GATATGAAGCAGCAGAATTTCTGTGTGG |
| P2 | PARP14 catalytic domain | AAGGGCATCGGTCGACTTATTATTTTCTAAA |
| | reverse | CGTAATAAGGTACTCTGGGTATGC |
| P3 | PARP 14 Macrodomain 2 | TTTCTATTGCTACAAACGCATACGCTATGCA |
| | forward | CCATCATCATCATCATATTGAGGG |
| P4 | PARP 14 Macrodomain 2 | CTCAAGGGCATCGGTCGACTTATTAACTAA |
| | reverse | CCAAATTGCCGTTTGCACG |
| P5 | pET28a reverse with Xba I site | TCTAGAAATAATTTTGTTTAACTTTAAGAAG |
| | | GAGATATACCATGCACCATCATCATCATCA |
| | | TATTGAGGGTC |
| P6 | pET28a forward with Xho I site | CTCGAGTTATTATTTTCTAAACGTAATAAGG |
| | for PARP14 catalytic domain | TACTCTGGG |
| P7 | pET28a forward with Xho I site | GTGGTGCTCGAGTTATTAAGCGTAATCTGGA |
| | for PARP14 Macrodomain 2 | ACATCGTATGGGTAACTGACGAGATTTCCA |
| | with a C-terminal HA tag | TTAGCCCTTC |
| P8 | PARP15 catalytic domain | CACCATCACCATCACATTGAAGGCCGTAAT |
| | forward | CTTCCTGAACACTGGACTGACATG |
| P9 | PARP15 catalytic domain | CTCAAGGGCATCGGTCGACTTATTAAGCCG |
| | reverse | TGAAAGTTATGAGATATTCTGGG |
| P10 | pET28a reverse with Xba I site | TCTAGAAATAATTTTGTTTAACTTTAAGAAG |
| | | GAGATATACCATGGGCCATCACCATCACCA |
| | | TCACATTG |
| P11 | pET28a forward with Xho I site | GTGGTGGTGGTGGTGCTCGAGTTATTAAGCC |
| | for PARP15 catalytic domain | GTGAAAGTTATGAGATATTCTGGG |

Macrodomain 2 (residue 999-1196) of PARP14 with an N-terminal His₆-tag and a Factor Xa cleavage site was amplified by PCR using primers P3 and P4, followed by incorporation of XhoI and XbaI restriction enzyme sites and a C-terminal HA-tag using primers P5 and P7 (Table 2). The amplified DNA fragments were digested by XhoI and XbaI restriction enzymes and then ligated into pET-28a(+) using T4 DNA ligase. All generated expression vectors were confirmed by DNA sequencing provided by Genewiz LLC (South Plainfield, N.J.).

BL21 (DE3) cells were transformed with the generated constructs for bacterial protein expression in LB Broth supplemented with kanamycin (50 μg mL⁻¹). The overnight bacterial culture (5 mL) was diluted into 1 liter LB Broth with kanamycin (50 pig mL⁻¹) for growth at 37° C. in an incubator shaker at speed of 250 rpm (Series 25, New Brunswick Scientific, N.J.). When OD₆₀₀ₙₘ reached 0.6-0.8, protein expression was induced with 0.5 mM isopropyl 3-D-1-thiogalactopyranoside (IPTG) for overnight at 22° C. Cells were then harvested by centrifugation at 4,550 g (Beckman J6B Centrifuge, JS-4.2 rotor), resuspended in equilibrium buffer (20 mM Tris-HCl, pH 8.0, 200 mM NaCl, 20 mM imidazole), and lysed using a French Press (GlenMills, N.J.) at 25,000 psi for three times. Cell debris was removed by centrifugation at 27,000 g for 1 hour (Beckman Coulter centrifuge, JA-17 rotor) and supernatant was filtered through a 0.45 μm membrane. The filtrate was loaded on a gravity flow column packed with 1 mL Ni-NTA agarose resin (Thermo Fisher Scientific, Waltham, Mass.), followed by washing with 15 column volumes of wash buffer (20 mM Tris-HCl, pH 8.0, 200 mM NaCl, 30 mM imidazole). Proteins were then eluted in 15 column volumes of elution buffer (20 mM Tris-HCl, pH 8.0, 200 mM NaCl, 400 mM imidazole), dialyzed in storage buffer (20 mM Tris-HCl, pH 8.0, 300 mM NaCl, 1 mM DTT, 10% glycerol) at 4° C. for overnight and another 6 hours in fresh storage buffer, and concentrated using an Amicon centrifugal concentrator (EMD Millipore, Temecula, Calif.) with a 10 kDa cutoff. Purified proteins were examined by SDS-PAGE and NanoDrop 2000° C. spectrophotometer (Thermo Fisher Scientific, Waltham, Mass.), and aliquoted and flash-frozen in liquid nitrogen for storage at −80° C. Calculated molecular extinction coefficient values are 1.20 for PARP 14, 1.13 for PARP15, and 0.93 for macrodomain 2.

### 2.3 MLISA Assay.

### 2.3.1 Overall Assay Design and Validation.

The general scheme of MLISA is shown in FIG. 1B. First, 200 μL of PARP-catalyzed automodifications using NAD⁺ as cosubstrate in reaction buffer (50 mM Tris-HCl, pH 7.4, 2 mM DTT) were performed in 96-wells plates together with the coating process for 2 hours at room temperature. Following five washes with 200 μL of PBST (0.1% v/v Tween-20 in PBS, pH 7.4) in each well, plates were blocked with 3% BSA dissolved in PBS, pH 7.4, for 2 hours under room temperature. Next, each well was washed five times using 200 μL of PBST and then incubated with 100 μL of purified macrodomain 2 (0.1 μM M2 for PARP15 and 0.3 M M2 for PARP14) in PBS for 1 hour at room temperature. Subsequently, plates were washed five times with 200 μL of PBST and incubated with 100 μL of anti-HA-HRP conjugate(1:5000 in PBS) for 1 hour at room temperature. After another five washes with 200 μL of PBST, 75 μL of QuantaBlu™ fluorogenic peroxidase substrate was added to each well and incubated for 10 min prior to reading. Fluorescence intensity in each well was then measured using Synergy H1 Multi-Mode Reader (BioTek, Winooski Vt.). All experiments were performed at least in triplicate.

Control wells were established to evaluate background signal intensity due to non-specific binding in between assay components. Wells for 2-hour PARP enzymatic reactions with 500 μM of NAD⁺ were set as the maximal signal wells while wells with only enzymes and no NAD⁺ addition were set to be the minimal signal wells. Fluorescence signal intensities of wells containing 3% BSA only, 3% BSA with various concentrations of M2, NAD⁺ plus various concentrations of M2, and enzymatic reactions without M2 addition were separately measured after incubation with anti-HA-HRP conjugate and compared to those of the maximal signal wells. MLISA assays for both PARP15 and PARP14 were performed on five plates with two sets of triplicates of the maximal and minimal signal wells on each plate for assay validation and repeatability purposes, of which three plates were carried out on the same day and two plates on different days. Assay quality was assessed through analyzing commonly accepted statistical parameters including signal-to-noise ratio (S/N), signal-to-background ratio (S/B) and screening window coefficient (Z′). (Venkannagari, H., et al. Eur. J Pharm. Sci. 49, 148-156, (2013)), (Zhang, J. H., et al. Journal of biomolecular screening 4, 67-73, (1999)); and (Iversen, P. W. et al. in Assay Guidance Manual (eds G. S. Sittampalam et al.) (Eli Lilly & Company and the National Center for Advancing Translational Sciences, 2004). Coefficients of variations (CVs) are calculated by analyzing all data on the assay plates and the normality of both maximal and minimal signal intensities of PARP15 and PARP14 was evaluated through Kolmogorov-Smirnov and D'Agostino and Pearson omnibus tests using Graphpad Prism (GraphPad Software, La Jolla, Calif.).

### 2.3.2 Time- and Concentration-Dependent PARP Catalytic Activities.

To perform activity assays with varied reaction time and concentrations, 96-well plates were coated with reaction mixtures in triplicates that consist of 500 μM of NAD⁺ and purified PARP enzymes at varied concentrations (PARP15: 500 nM, 1 SAM, and 2 μM; PARP14: 3 μM, 6 μM, and 9 μM) in reaction buffer (50 mM Tris-HCl, pH 7.4, 2 mM DTT) and incubated for different time points (PARP15: 5, 10, 20, 30, 40, 50 and 60 minutes; PARP14: 0.5, 1, 2, 3, 4, 5 and 6 hours) at room temperature. The reactions were quenched with 20% ice-cold TCA at those time points. Background signals (from no NAD⁺ wells) were subtracted from the reaction wells for baseline correction purposes using Graphpad Prism.

### 2.3.3 Inhibition of PARP Enzymatic Activities.

To carry out inhibition assays, 96-well plates were coated with reactions in triplicates that consist of 500 μM of NAD⁺, purified PARP enzymes (PARP15: 500 nM; PARP14: 3 μM), and various inhibitors at multiple concentrations (0, 0.5, 1, 2.5, 5, 7.5, 10, 12.5, and 15 μM for PARP15, 0.1% DMSO or water and 0, 1, 2.5, 5, 10, 25, 50, 75, and 100 μM for PARP14, 0.6% DMSO or water) in reaction buffer (50 mM Tris-HCl, pH 7.4, 2 mM DTT). Inhibitors were initially dissolved in 100% DMSO and diluted in water to reduce DMSO content to either 0.1% (for PARP15) or 0.6% (for PARP14) in each well. Wells without inhibitors and with 0.1% DMSO (PARP15) or 0.6% DMSO (PARP14) were included as separate controls based on the solubility of inhibitors. IC₅₀ and pIC₅₀ values of individual inhibitors were calculated by fitting the dose-response curves with four parameters in Graphpad Prism for both PARP15 and PARP14 (Selvaraj, C., et al. Current Trends in Biotechnology & Pharmacy 5 (2011)).

### 2.3.4 Enzyme Kinetics of PARP1S-Catalyzed Automodification.

To characterize enzyme kinetics, 96-well plates were coated with reactions in triplicates that consist of 500 nM of PARP15 and NAD⁺ at varied concentrations (5, 10, 20, 30, 40, 200, and 400 μM) in reaction buffer (50 mM Tris-HCl, pH 7.4, 2 mM DTT). The reactions were quenched with 20% ice-cold TCA at different time points (0, 2.5, 5, 7.5, 10, 12.5, 15, and 20 minutes). The plates were further incubated for up to 2 hours under room temperature followed by five washes with 200 μL of PBST. Kinetic parameters were calculated by fitting data to Michaelis-Menten model implemented in GraphPad Prism.

To determine reaction rates, standard curves were generated for each reaction plate. In brief, PARP15-catalyzed automodification reactions were incubated for 2 hours under room temperature in 1.5 mL microcentrifuge tubes that contained 50 μg mL⁻¹ of purified PARP15 and 500 μM of NAD⁺ in reaction buffer (50 mM Tris-HCl, pH 7.4, 2 mM DTT). Reaction mixtures were then serially diluted and plated simultaneously on both the 96-well ELISA plates and clear 96-well pureGrade plates. Upon additions of Coomassie Plus (Bradford) assay reagents to clear 96-well pureGrade plates, the absorbance at 595 nm of each well was measured by Synergy H1 Hybrid Multi-Mode Microplate Reader. The concentrations of automodified PARP15 were calculated through fitting to linear regression curves generated with BSA standards. Standard curves were constructed through linear correlation of the determined concentrations of automodified PARP15 with the fluorescence intensities of the corresponding wells measured on 96-well ELISA plates.

### 2.4 High-Performance Liquid Chromatography (HPLC)-Based Activity Assay.

PARP-catalyzed automodification reactions were performed at room temperature in 100 μL assay solutions containing 50 mM Tris-HCl, pH 7.4, 2 mM DTT, and varied concentrations of NAD⁺ and purified PARP enzymes. The reaction mixtures after varied lengths of incubation were separated by reverse phase HPLC using a semipreparative C18 Kinetex® column (5 μm, 100 Å, 150×10.0 mm, Phenomenex Inc, Torrance, Calif.) with a gradient of methanol (0-50% in 12 min) in water containing 0.1% formic acid. Reaction rates were determined on the basis of the assigned peaks of nicotinamide and NAD⁺.

### 3. Results

### 3.1 Overall Assay Design.

Macrodomain 2 of PARP14 was chosen for developing the MLISA, since it shows high affinity to mono-ADP-ribose, but displays no hydrolase activity seen in many other macrodomain proteins (Rosenthal, F. et al. Nat. Struct. Mol. Biol. 20, 502-507, (2013), Forst, A. H. et al. Structure 21, 462-475, (2013)). The bacterial expression construct for macrodomain 2 was designed with an N-terminal His₆ tag followed by a Factor Xa cleavage site for purification and a C-terminal HA tag for recognition by a secondary antibody. Recombinant macrodomain 2 was stably expressed in E. coli and purified by Ni-NTA affinity chromatography with a final yield of 1.4 mg per liter. SDS-PAGE revealed that the generated macrodomain 2 migrated as a single band around 24 kDa (FIG. 1C).

Next, to test the generality of the resulting macrodomain 2 in binding and reporting mono-ADP-ribosylation, two human mono-ARTs PARP15 and PARP14 were recombinantly produced. Overexpression of these mono-ARTs are frequently detected in many types of cancer, but little is known about the characteristics of these enzymes (Andersson, C. D. et al. J. Med. Chem. 55, 7706-7718, (2012)). All PARPs were stably expressed in E. coli and purified using the same method as macrodomain 2 with final yields of 1-1.5 mg per liter. SDS-PAGE showed single bands of 25 kDa for PARP15 and 23 kDa for PARP14 (FIG. 1C). The catalytic activities of the generated PARPs were verified through automodification reactions using HPLC-based activity assay.

The MLISA was designed by utilizing the HA-tagged macrodomain 2 as a primary detection agent for recognition of mono-ADP-ribosylated proteins that are immobilized on ELISA plates (FIG. 1B). An anti-HA antibody-HRP conjugate was included as a dual agent for detecting bound macrodomain 2 and reporting levels of activity through enzyme-mediated signal amplification. First, automodification reactions by mono-ARTs were performed in the wells for direct coating of ADP-ribosylated proteins on 96-well plates. The reactions could be quenched by adding 20% TCA to each well. By dispensing libraries of compounds onto the plates, their effects on mono-ART activities could be quantitatively measured for identification of new activators/inhibitors. Second, 3% BSA in PBS pH 7.4 was utilized as blocking agent following the coating step, similar to conventional immunoassays. Then, HA-tagged macrodomain 2 and anti-HA antibody-HRP conjugate were added to each well in a sequential order for complex assembly. It should also be noted that each of these steps was incubated for 1-2 hours at room temperature and thorough washes (5×) with PBST were carried out prior to additions of any reagents for next step. Lastly, upon addition of fluorogenic substrates of HRP to each well, enzymatic activities were determined on the basis of recorded fluorescence intensities that closely correlate with the levels of immuno-complexes formed in the wells.

### 3.2 Validation of MLISA.

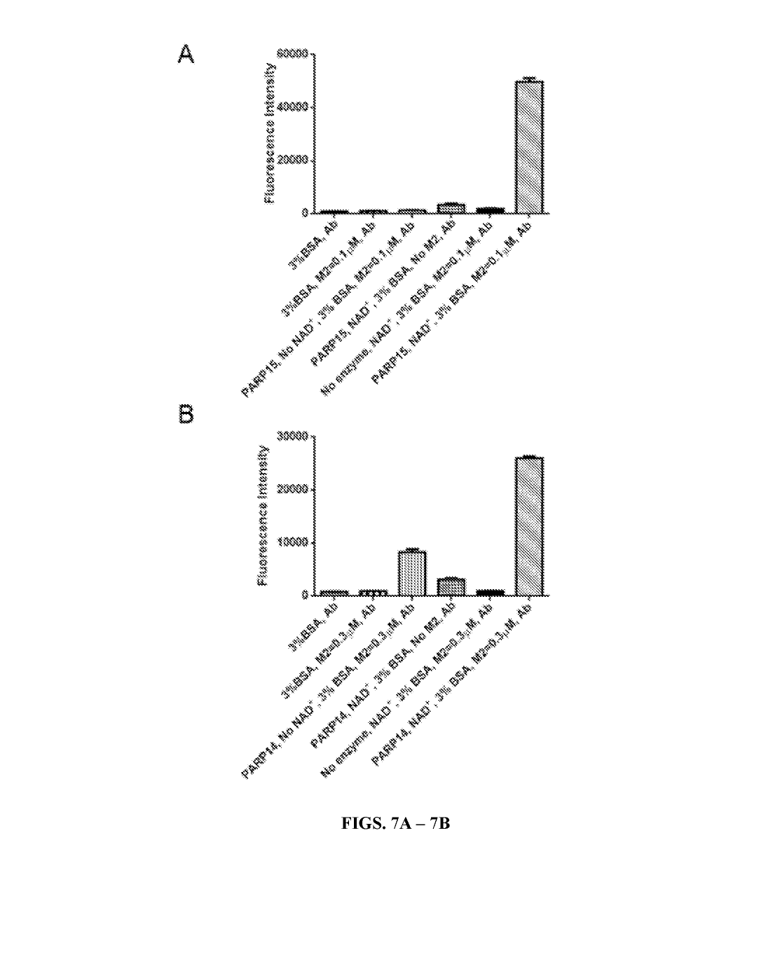

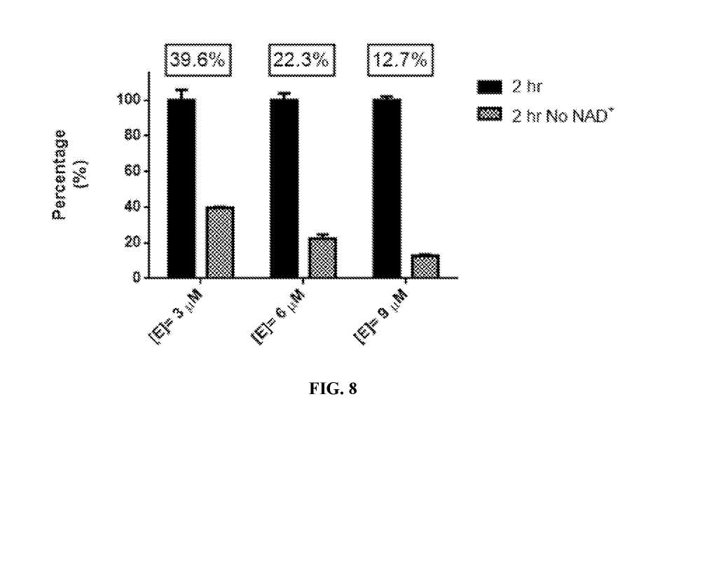

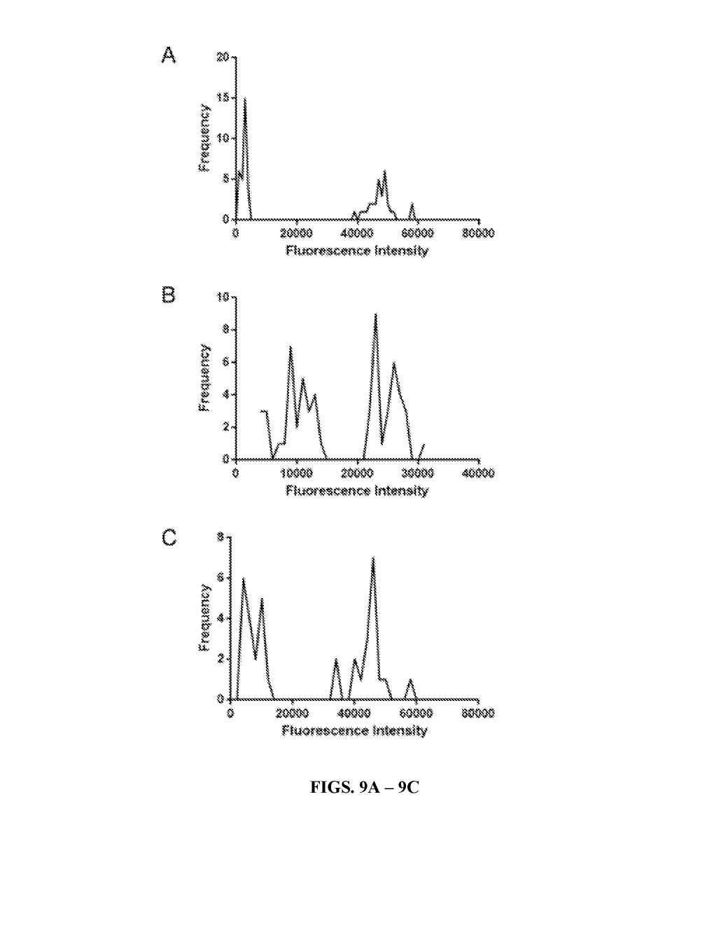

By performing MLISA with auto-ADP-ribosylation catalyzed by different mono-ARTs, the specificity of macrodomain 2 towards mono ADP-ribosylated proteins was examined. Reactions containing PARP enzymes with and without NAD⁺ were incubated for two hours in the wells, followed by blocking with 3% BSA, detecting with macrodomain 2, and the reporting step as established for the MLISA. In comparison to the control wells where no reactions occur or no macrodomain 2 existed, the respective ones with NAD⁺-dependent auto-ADP-ribosylation showed dramatically increased fluorescence intensities for both PARPs (FIGS. 7A-7B). This indicated that the generated macrodomain 2 binds specifically to mono-ADP-ribosylated proteins. Using macrodomain 2 as a detection agent of mono-ADP-ribose, MLISA allows quantitative measurements of mono-ADP-ribosylation on distinct proteins. It was found that relative to reaction wells, the control wells with PARP14 revealed higher background fluorescence intensities than those with PARP15, likely resulting from the nonspecific binding caused by high concentration of PARP14 (3 AM for PARP14 and 500 nM for PARP15). Importantly, by increasing the concentrations of PARP14 to 6 and 9 μM for enhanced mono-ADP-ribosylation, lower contributions of fluorescence signals from the background were observed (FIG. 8), supporting high specificity of the generated macrodomain 2 for mono-ADP-ribosylated proteins. Relative to PARP15, higher macrodomain 2 concentration (0.3 μM versus 0.1 μM) was used for PARP14 to improve signal-to-background ratio (>2.5). Additions of anti-HA antibody-HRP conjugate with and without macrodomain 2 to the wells with only 3% BSA resulted in minimal fluorescence intensity, showing that neither of these reagents binds nonspecifically to BSA. In the absence of macrodomain 2, incubation of anti-HA antibody-HRP conjugate with PARP-catalyzed reactions led to low fluorescence signals, indicating the lack of specific binding to ADP-ribosylated proteins for the anti-HA secondary antibody. Compared with reaction wells, the control wells without PARP enzymes gave minimal fluorescence signals, showing no affinity between macrodomain 2 and NAD⁺. Taken together, these results support the use of recombinant macrodomain 2 in MLISA as a specific detection agent for mono-ADP-ribosylated proteins. The well-to-well, plate-to-plate, and day-to-day variations and frequency distribution of maximal and minimal signals were evaluated through analysis of five independent assay plates performed on different days (Tables 4 and 3; FIG. 9).

TABLE 3
Evaluation of % CV¹ of maximal and
minimal signals using SD² criteria.

| | PARP15 | PARP14 |
|---|---|---|
| SDₘₐₓ/SDₘᵢₙ, day to day | 3673/840 | 1699/1615 |
| SDₘₐₓ/SDₘᵢₙ, well to well | 4031/902 | 2183/2940 |
| SDₘₐₓ/SDₘᵢₙ, plate to plate | 2631/289 | 1730/270 |

¹CV: coefficient of variation
²SD: standard deviation

The Z′ factors for PARP 15- and PARP14-catalyzed automodifications are 0.8 and 0.6, respectively, indicating that the developed MLISA assay is suitable for screening purposes.

TABLE 4
Statistical parameters of optimized
MLISA for PARP15 and PARP14.

| | PARP15 | PARP14 |
|---|---|---|
| S/B¹ | 19.9 ± 3.4 | 2.7 ± 0.7 |
| S/N² | 12.4 ± 1.7 | 6.3 ± 0.2 |
| Z′³ | 0.8 ± 0.1 | 0.6 ± 0.1 |
| Day to day, CV⁴ | 7.6 ± 1.0/37.1 ± 22.4 | 6.8 ± 0.7/18.8 ± 11.1 |
| (max/min; %) | | |
| Well to well, CV | 8.5/35.7 | 8.7/31.0 |
| (max/min; %) | | |
| Plate to plate, CV | 5.5 ± 0.9/11.4 ± 4.5 | 6.9 ± 1.1/2.8 ± 1.1 |
| (max/min; %) | | |

¹S/B: Signal to Background ratio.
²S/N: Signal to Noise ratio.
³Z′: Z factor.
⁴CV: Coefficient of variation

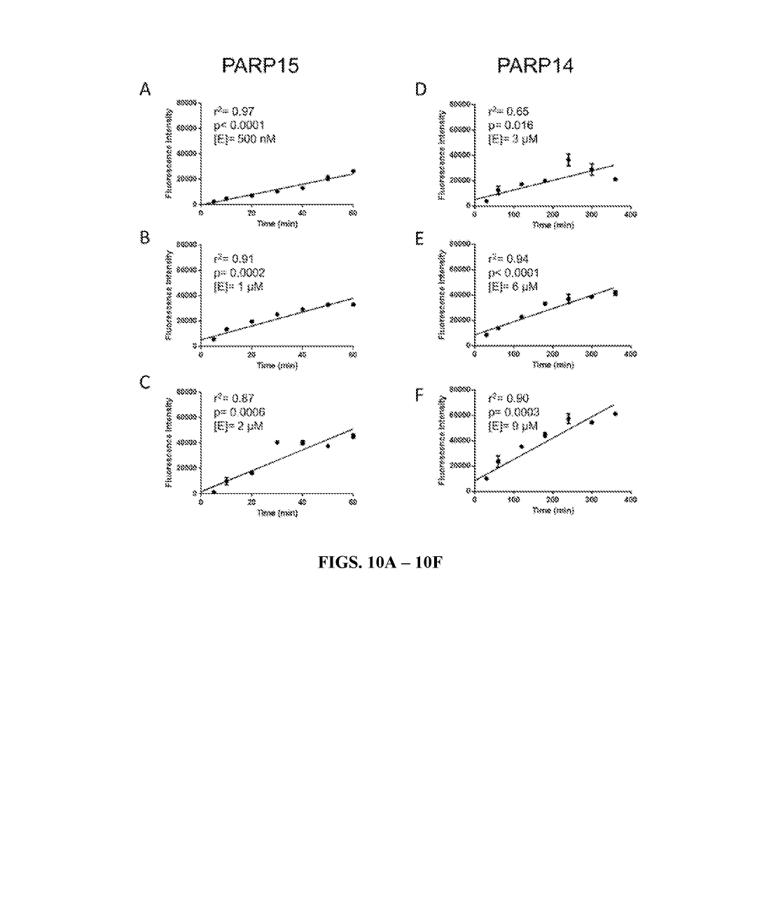

Next, catalytic activities of PARP15 and PARP14 were characterized by MLISA at varied lengths of reaction times and different enzyme concentrations. Automodification of PARP15 was incubated for 5, 10, 20, 30, 40, 50, and 60 minutes at 500 nM, 1 μM, and 2 μM. Upon corrections by background signals (from no NAD⁺ wells), the reaction wells showed that the fluorescence intensities for PARP15-catalyzed automodification reactions increased in concentration- and time-dependent manners (FIGS. 10A-10C), allowing quantitative measurements of PARP15 activities by MLISA. Similarly, the concentration- and time-dependent increases in fluorescence signals were seen for PARP14-catalyzed automodifications which were carried out at both higher enzyme concentrations and longer time duration (FIGS. 10D-10F). These data indicated that PARP14 enzymatic activity can also be quantitatively determined by MLISA. The time-dependent increases in PARP activities were more significant at higher enzyme concentrations, possibly due to weaker activities at lower concentrations of enzymes. Collectively, MLISA is shown as a general method for qualitative and quantitative characterization of mono-ARTs.

### 3.3 Characterization of Enzyme Kinetics by MLISA.

The developed MLISA was then utilized to characterize enzyme kinetics of mono-ARTs. PARP15 was selected as a model enzyme since a published Km value for its automodification reaction was available (Karlberg, T. et al. J. Biol. Chem. 290, 7336-7344, (2015)). MLISA-based PARP15-catalyzed automodifications were performed with NAD⁺ at varied concentrations. The enzymatic reactions were quenched with ice cold 20% TCA at various time points. As described in the experimental methods, standard curves were created on each plate for determining the concentrations of generated automodified PARP15 on the basis of measured fluorescence intensities. By fitting the kinetic data to Michaelis-Menten equation, the k_{cat} and Kₘ of PARP15 for automodification were calculated to be 0.011±0.001 min⁻¹ and 4.5±2.9 μM (FIG. 4), respectively, which is consistent with the Km value of 5.8±1.9 μM as reported previously (Karlberg, T. et al. J. Biol. Chem. 290, 7336-7344, (2015)). This supports the use of MLISA as a direct method for examining kinetics of mono-ARTs. It was noted that determination of the reaction rates with NAD⁺<5 M is difficult, due to low fluorescence signals over the background and relatively large variations.

### 3.4 Evaluation of PARP Inhibitors by MLISA.

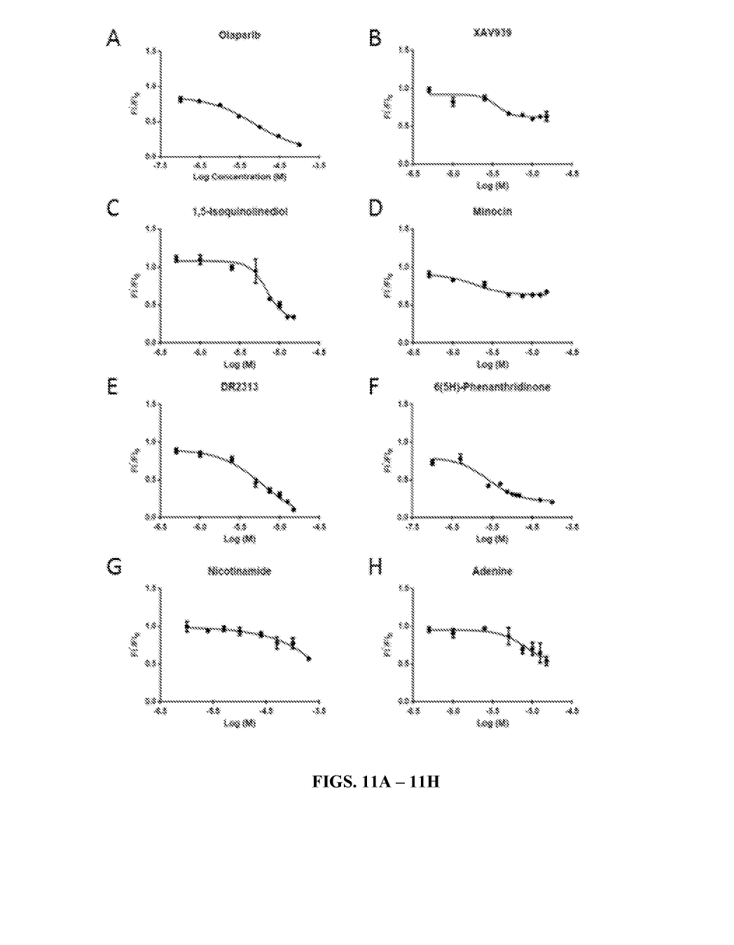

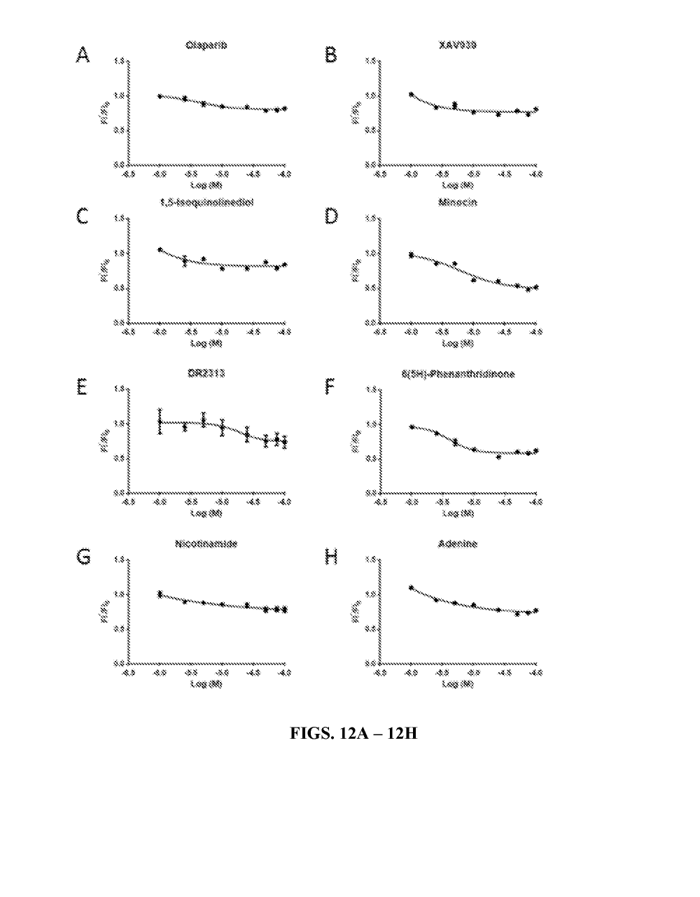

Applicant next applied the MLISA for inhibitor screening of PARP15 and PARP14, given that both mono-ART enzymes are involved in many human diseases including cancer (Cho, S. H. et al. Blood 113, 2416-2425, (2009), (Di Paola, S., et al. PloS one 7, (2012), (Gariani, K. et al. J. Hepatol., (2016), (Iansante, V. et al. Nat. Commun. 6, 7882, (2015), (Jwa, M. & Chang, Nat. Cell Biol. 14, 1223-1230, (2012), (Mehrotra, P. et al. J. Allergy Clin. Immunol. 131, 521-531 (2013), (Riffell, J. L., et al. Nat. Rev. DrugDiscov. 11, 923-936, (2012), (Ryu, D. et al. Sci. Transl. Med. 8, 361ra139, (2016), (Vyas, S. & Chang, Nat. Rev. Cancer 14, 502-509, (2014), (Welsby, I., et al. Biochem. Pharmacol. 84, 11-20, (2012), (Ekblad, T. et al. Eur. J. Med. Chem. 95, 546-551, (2015)). A panel of eight commonly used chemical tools for PARPs was examined in 96-well plates for their inhibitory effects on automodifcations of PARP15 and PARP14 at different concentrations (Table 1). Since some of those compounds were tested in 0.1% or 0.6% DMSO, control wells containing 0.1% or 0.6% DMSO only were used as no inhibitor controls to even out the inhibitory effect of DMSO on PARP enzymes. It was found that several inhibitors exhibit dose-dependent inhibition on catalytic activities of PARP15 with IC₅₀ values in the range of 2-8 μM (Table 5 and FIG. 11). Both Olaparib and 6(5H)-phenanthridinone display moderate inhibition activity for PARP15 with determined IC₅₀ values of 7.3±1.3 and 2.4±0.6 μM, respectively, which are consistent with previous studies (Venkannagari, H., et al. Eur. J. Pharm. Sci. 49, 148-156, (2013), (Wahlberg, E. et al. Nat. Biotechnol. 30, 283-288, (2012)). Similarly, 1, 5-isoquinolinediol and DR2313 were found to inhibit PARP15 activity with IC₅₀ of 6.9±0.7 and 6.3±1.7 μM, respectively. The other four compounds including XAV939, minocin, nicotinamide, and adenine gave no significant inhibition effects on PARP15 activity at concentrations up to 100 μM. In contrast to PARP15, none of the eight tested compounds revealed dose-dependent inhibition activity against PARP14 at concentrations up to 100 μM (FIG. 12), suggesting large differences in the active sites and/or catalytic mechanisms of PARP15 and PARP14. It was shown that MLISA offers a direct and convenient approach for characterization of mono-ART modulators, which is likely to be suitable for high-throughput screening.

### 4. Discussion

Reported herein is an innovative MLISA assay for studying mono-ARTs by exploiting the macrodomain protein as a binding module of mono-ADP-ribose. As a general approach, it may be applicable to investigation of various types of mono-ART enzymes and qualitative and quantitative characterization of mono-ART activities and their modulators. Similar to conventional ELISA, the developed MLISA can possibly be performed in different formats through uses of modified and/or new reagents, including direct, sandwich, and competitive manners. The versatility in assay style would further expand its applications in identifying readers and erasers of mono-ADP-ribosylation and examining enzyme-specific modifications. Through the use of a secondary antibody-HRP conjugate to amplify levels of modifications detected by the macrodomain protein, MLISA is characterized by a wide range of signal intensity, allowing measurements of rapid turnovers of NAD⁺ by mono-ARTs and identification of enzyme inhibitors. Additionally, MLISA requires no radioactive NAD⁺ or specialized NAD⁺ analogues and utilizes reagents which need no special handling and are readily accessible, providing a relatively low-cost and high-accessibility approach for evaluating mono-ART enzymes. By directly measuring and reporting levels of enzyme activities, MLISA reduces complexity of the reaction systems and minimizes experimental variations.

TABLE 5
Inhibitory potency of eight compounds for PARP15.

| Compound | PARP15 IC₅₀ (μM) | CI¹ (μM) | pIC₅₀ |
|---|---|---|---|
| Olaparib | 7.3 ± 1.3 | 6.0 to 8.6 | 5.1 |
| XAV939 | >15 μM | N.D.² | N.D. |
| 1,5-Isoquinolinediol | 6.9 ± 0.7 | 6.2 to 7.6 | 5.2 |
| Minocin | >15 μM | N.D. | N.D. |
| DR2313 | 6.3 ± 1.7 | 4.6 to 8.0 | 5.2 |
| 6(5H)-Phenanthridinone | 2.4 ± 0.6 | 1.8 to 3.0 | 5.6 |
| Nicotinamide | >15 μM | N.D. | N.D. |
| Adenine | >15 μM | N.D. | N.D. |

¹CI: 95% Confidence Intervals.
²N.D.: not determined

Significant levels of fluorescence intensity were observed in MLISA for the control wells with 3 μM PARP14 enzyme only, suggesting that in the absence of ADP-ribosylation the recombinant macrodomain 2 binds to catalytic domain of PARP14. It is likely that the macrodomain 2 of PARP14, which is a multidomain protein with a molecular weight of approximately 203 kDa (Hakme, A., et al. Dev. Dyn. 237, 209-215, (2008), can form interactions with the catalytic domain within the protein architecture to coordinate or regulate biological functions. When recombinantly produced, these subdomains of PARP14 may still interact with each other, causing higher background signals as observed in the wells with PARP14 enzyme only.

Olaparib, a potent inhibitor of poly-ARTs, showed less inhibitory activity against mono-ARTs comparing to PARP1 (IC₅₀=5 nM⁵⁶), consistent with previous studies(Venkannagari, H., et al. Eur. J. Pharm. Sci. 49, 148-156, (2013), (Wahlberg, E. et al. Nat. Biotechnol. 30, 283-288, (2012)). The preferential binding of olaparib to poly-ARTs possibly resulted from differences in catalytic mechanisms, overall structural folds, catalytic elements, and active site interactions. Extensive mechanistic and structural studies have been performed with poly-ARTs with diverse groups of inhibitors, which facilitate elucidation of principles underlying the catalysis and inhibition of poly-ARTs. In comparison, limited information is available for selective inhibition of mono-ARTs (Ekblad, T. et al. Eur. J. Med. Chem. 95, 546-551, (2015)). In this study, several compounds including olaparib revealed differential inhibition effects on catalytic activities of PARP15 and PARP14. This suggested that new inhibitors specific for individual mono-ARTs could possibly be identified through mechanism and/or structure-based rational design or compound library-based screening. In fact, a potent PARP14 inhibitor was recently identified using a small molecule microarray, which exhibits more than 20-fold selectivity over PARP1 (Peng, B., et al. Angew. Chem. Int. Ed. Engl. 56, 248-253, (2017)).

In addition to macrodomain 2 of PARP14, other macrodomains in the superfamily can possibly be utilized as detection agents for the ADP-ribose moiety, depending on their binding affinities, specificity, and capabilities in hydrolysis. For example, macrodomain 3 of PARP14 was also shown to bind tightly to mono-ADP-ribose in vitro (Feijs, K. L. H., et al. Nat. Rev. Mol. Cell Biol. 14, 443-451, (2013), (Forst, A. H. et al. Structure 21, 462-475, (2013)). Similarly, PARP15 is a macrodomain-containing protein. In contrast to these mono-ADP-ribose binding modules, macrodomain proteins recognizing poly-ADP-ribose units have been identified, such as PARP9 and Af1521 (Karras, G. I. et al. EMBO J. 24, 1911-1920, (2005), suggesting the possibility of characterizing poly-ARTs with macrodomains. Indeed, macrodomains have been utilized to identify and visualize cellular ADP-ribosylated proteins (Aguilera-Gomez, A., et al. eLife 5, (2016), (Dani, N. et al. Proc. Natl. Acad. Sci. U.S.A. 106, 4243-4248, (2009), (Forst, A. H. et al. Structure 21, 462-475, (2013), (Vivelo, C. A., et al. Proteomics 15, 203-217, (2015)). It is of note that some of the macrodomain proteins were found to have ADP-ribosylhydrolase activities including Af1521, human MacroD1, MacroD2, and C6orf130, preventing them from use in detecting protein ADP-ribosylation (Rosenthal, F. et al. Nat. Struct. Mol. Biol. 20, 502-507, (2013), (Jankevicius, G. et al. Nat. Struct. Mol. Biol. 20, 508-514, (2013)). Besides macrodomains, recent studies discovered that WWE domains can uniquely recognize poly ADP-ribose units (Zhang, Y. et al. Nat. Cell Biol. 13, 623-629, (2011), (Kang, H. C. et al. Proc. Natl. Acad. Sci. U.S.A. 108, 14103-14108, (2011), (Wang, Z. et al. Genes Dev. 26, 235-240, (2012), (Gibson, B. A., et al. Nat. Rev. Mol. Cell Biol. 13, 411-424 (2012), representing a new class of protein tools for studying protein ADP-ribosylation. Moreover, the high specificity of macrodomains and WWE domains for ADP-ribose possibly allow studies of extracellular ADP-ribosylation by distinct ART enzymes. Guided by X-ray crystal structures of various macrodomains, protein engineering can be performed to create variants with improved affinity and specificity or orthogonal pairs of macrodomain and non-canonical ADP-ribose. The resulting engineered macrodomains may provide important tools for investigating protein ADP-ribosylation and modulating signaling pathways and biological processes regulated by macrodomains.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Sequence Listing

M2 DNA sequence
atgcaccatcatcatcatcatattgagggtcgcgggaaaacatcatgggaaaaaggaagcctggtgtccccgggaggcctgcagatg
ctgttggtgaaagagggtgtgcagaatgctaagaccgatgttgttgtcaactccgttcccttggatctcgtgcttagtagagggcctctttc
taagtccctcttggaaaaagctggaccagagctccaggaggaattggacacagttggacaaggggtggctgtcagcatgggcacagt
gctcaaaaccagcagctggaatctggactgtcgctatgtgcttcacgtggtagctccggagtggagaaatggtagcacatcttcactca
agataatggaagacataatcagagaatgtatggagatcactgagagcttgtccttaaaatcaattgcatttccagcaataggaacaggaa
acttgggatttcctaaaaacatattcgctgaattaatcatttcagaggtgttcaaatttagtagcaagaatcagctgaaaactttacaagagg
ttcactttctgctgcacccgagtgatcatgaaaatattcaggcattttcagatgaatttgccagaagggctaatggaaatctcgtcagttac
ccatacgatgttccagattacgct
M2 protein sequence
MHHHHHHIEGRGKTSWEKGSLVSPGGLQMLLVKEGVQNAKTDVVVNSVPLDLVLS
RGPLSKSLLEKAGPELQEELDTVGQGVAVSMGTVLKTSSWNLDCRYVLHVVAPEW
RNGSTSSLKIMEDIIRECMEITESLSLKSIAFPAIGTGNLGFPKNIFAELIISEVFKFSSKN
QLKTLQEVHFLLHPSDHENIQAFSDEFARRANGNLVSYPYDVPDYA
PARP9 protein sequence
MHHHHHHIEGRIQQQKTQDEMKENIIFLKCPVPPTQELLDQKKQFEKCGLQVLKVEK
IDNEVLMAAFQRKKKMMEEKLHRQPVSHRLFQQVPYQFCNVVCRVGFQRMYSTPC
DPKYGAGIYFTKNLKNLAEKAKKISAADKLIYVFEAEVLTGFFCQGHPLNIVPPPLSP
GAIDGHDSVVDNVSSPETFVIFSGMQAIPQYLWTCTQEYVQSQDYSSGPMRPFAQHP
WRGFASG
PARP14 DNA sequence
atgcaccatcatcatcatcatattgagggtcgcgatatgaagcagcagaatttctgtgtggtggagctgctgcctagtgatcctgagtaca
acacggtggcaagcaagtttaatcagacctgctcacacttcagaatagagaagattgagaggatccagaatccagatctctggaatag
ctaccaggcaaagaaaaaaactatggatgccaagaatggccagacaatgaatgagaagcaactcttccatgggacagatgccggct
ccgtgccacacgtcaatcgaaatggctttaaccgcagctatgccggaaagaatgctgtggcatatggaaagggaacctattttgctgtc
aatgccaattattctgccaatgatacgtactccagaccagatgcaaatgggagaaagcatgtgtattatgtgcgagtacttactggaatct
atacacatggaaatcattcattaattgtgcctccttcaaagaaccctcaaaatcctactgacctgtatgacactgtcacagataatgtgcac
catccaagtttatttgtggcattttatgactaccaagcatacccagagtaccttattacgtttagaaaa
PARP14 protein sequence
MHHHHHHIEGRDMKQQNFCVVELLPSDPEYNTVASKFNQTCSHFRIEKIERIQNPDL
WNSYQAKKKTMDAKNGQTMNEKQLFHGTDAGSVPHVNRNGFNRSYAGKNAVAY
GKGTYFAVNANYSANDTYSRPDANGRKHVYYVRVLTGIYTHGNHSLIVPPSKNPQN
PTDLYDTVTDNVHHPSLFVAFYDYQAYPEYLITFRK
PARP15 DNA sequence
atgggccatcaccatcaccatcacattgaaggccgtaatcttcctgaacactggactgacatgaatcatcagctgttttgcatggtccagc
tagagccaggacaatcagaatataataccataaaggacaagttcacccgaacttgttcttcctacgcaatagagaagattgagaggata
cagaatgcatttctctggcagagctaccaggtaaagaaaaggcaaatggatatcaagaatgaccataagaataatgagagactcctctt
ccatgggacagatgcagactcagtgccatatgtcaatcagcacggctttaatagaagttgtgctgggaaaaatgctgtatcctatggaaa
aggaacctattttgctgtggatgccagttattctgccaaggacacctactccaagccagacagcaatgggagaaagcacatgtacgttg
tgcgagtacttactggagtcttcacaaagggacgtgcaggattagtcacccctccacccaagaatcctcacaatcccacagatctctttg
actcagtgacaaacaatacacgatctccaaagctatttgtggtattctttgataatcaggcttacccagaatatctcataactttcacggct
PARP15 Protein sequence
MGHHHHHHIEGRNLPEHWTDMNHQLFCMVQLEPGQSEYNTIKDKFTRTCSSYAIEKI
ERIQNAFLWQSYQVKKRQMDIKNDHKNNERLLFHGTDADSVPYVNQHGFNRSCAG
KNAVSYGKGTYFAVDASYSAKDTYSKPDSNGRKHMYVVRVLTGVFTKGRAGLVTP
PPKNPHNPTDLFDSVTNNTRSPKLFVVFFDNQAYPEYLITFTA
PARP16 DNA sequence
atgggccatcaccatcaccatcacattgaaggccgtggctgggcggccgccagggaggcggcgggccgcgacatgctggccgcc
gacctccggtgcagcctcttcgcctcggccctgcagagctacaagcgcgactcggtgctgcggcccttccccgcgtcctacgcccgc
ggcgactgtaaggactttgaagccctgcttgcagatgccagcaagttacctaacctgaaagaacttctccagtcctccggagacaacc
acaaacgggcctgggacctggtgagctggattttatcctcaaaggtcctgacaatccacagtgcagggaaggcagagtttgaaaagat
ccaaaagctgactggggctcctcacacgcctgttcctgcaccggacttcctgtttgaaattgagtactttgacccagccaacgccaaattt
tatgagaccaaaggagaacgagacctaatctatgcatttcatggtagccgcctagaaaacttccattccattatccacaatggcctgcac
tgccatctgaacaagacatccttgttcggagaggggacctacctcaccagtgacttaagcctggccctcatatacagcccccatggcca
tgggtggcagcacagcctcctcggccccatccttagctgtgtggccgtgtgtgaggtcattgaccatccggacgtcaagtgccaaacc
aagaagaaggattccaaggagatagatcgcagacgagcgagaatcaaacatagtgaagggggagacatccctcccaagtacttcgt
ggtcaccaataaccagctgctgcgagtgaagtacctcctggtgtattcacagaagccacccaagagggct
PARP16 protein
MGHHHHHHIEGRGWAAAREAAGRDMLAADLRCSLFASALQSYKRDSVLRPFPASY
ARGDCKDFEALLADASKLPNLKELLQSSGDNHKRAWDLVSWILSSKVLTIHSAGKA
EFEKIQKLTGAPHTPVPAPDFLFEIEYFDPANAKFYETKGERDLIYAFHGSRLENFHSII
HNGLHCHLNKTSLFGEGTYLTSDLSLALIYSPHGHGWQHSLLGPILSCVAVCEVIDHP
DVKCQTKKKDSKEIDRRRARIKHSEGGDIPPKYFVVTNNQLLRVKYLLVYSQKPPKR
A
PARP14 Macrodomain2 vector
atccggatatagttcctcctttcagcaaaaaacccctcaagacccgtttagaggccccaaggggttatgctagttattgctcagcggtgg
cagcagccaactcagcttcctttcgggctttgttagcagccggatctcagtggtggtggtggtggtgctcgagttattaAGCGTAA

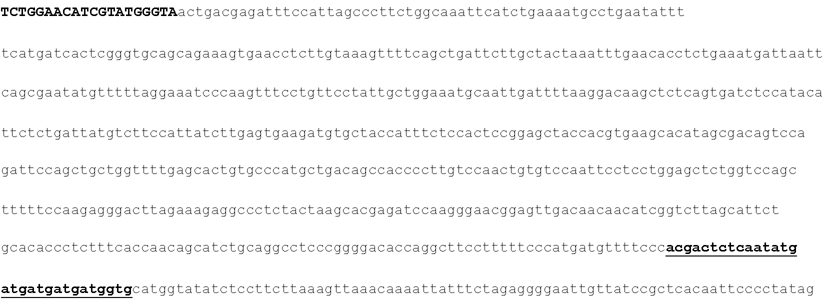

tgagtcgtattaatttcgcgggatcgagatctcgatcctctacgccggacgcatcgtggccggcatcaccggcgccacaggtgcggtt
gctggcgcctatatcgccgacatcaccgatggggaagatcgggctcgccacttcgggctcatgagcgcttgtttcggcgtgggtatgg
tggcaggccccgtggccgggggactgttgggcgccatctccttgcatgcaccattccttgcggcggcggtgctcaacggcctcaacc
tactactgggctgcttcctaatgcaggagtcgcataagggagagcgtcgagatcccggacaccatcgaatggcgcaaaacctttcgc
ggtatggcatgatagcgcccggaagagagtcaattcagggtggtgaatgtgaaaccagtaacgttatacgatgtcgcagagtatgccg
gtgtctcttatcagaccgtttcccgcgtggtgaaccaggccagccacgtttctgcgaaaacgcgggaaaaagtggaagcggcgatgg
cggagctgaattacattcccaaccgcgtggcacaacaactggcgggcaaacagtcgttgctgattggcgttgccacctccagtctggc
cctgcacgcgccgtcgcaaattgtcgcggcgattaaatctcgcgccgatcaactgggtgccagcgtggtggtgtcgatggtagaacg
aagcggcgtcgaagcctgtaaagcggcggtgcacaatcttctcgcgcaacgcgtcagtgggctgatcattaactatccgctggatgac
caggatgccattgctgtggaagctgcctgcactaatgttccggcgttatttcttgatgtctctgaccagacacccatcaacagtattattttc
tcccatgaagacggtacgcgactgggcgtggagcatctggtcgcattgggtcaccagcaaatcgcgctgttagcgggcccattaagtt
ctgtctcggcgcgtctgcgtctggctggctggcataaatatctcactcgcaatcaaattcagccgatagcggaacgggaaggcgactg
gagtgccatgtccggttttcaacaaaccatgcaaatgctgaatgagggcatcgttcccactgcgatgctggttgccaacgatcagatgg
cgctgggcgcaatgcgcgccattaccgagtccgggctgcgcgttggtgcggatatctcggtagtgggatacgacgataccgaagac
agctcatgttatatcccgccgttaaccaccatcaaacaggattttcgcctgctggggcaaaccagcgtggaccgcttgctgcaactctct
cagggccaggcggtgaagggcaatcagctgttgcccgtctcactggtgaaaagaaaaaccaccctggcgcccaatacgcaaaccg
cctctccccgcgcgttggccgattcattaatgcagctggcacgacaggtttcccgactggaaagcgggcagtgagcgcaacgcaatt
aatgtaagttagctcactcattaggcaccgggatctcgaccgatgcccttgagagccttcaacccagtcagctccttccggtgggcgcg
gggcatgactatcgtcgccgcacttatgactgtcttctttatcatgcaactcgtaggacaggtgccggcagcgctctgggtcattttcggc
gaggaccgctttcgctggagcgcgacgatgatcggcctgtcgcttgcggtattcggaatcttgcacgccctcgctcaagccttcgtcac
tggtcccgccaccaaacgtttcggcgagaagcaggccattatcgccggcatggcggccccacgggtgcgcatgatcgtgctcctgtc
gttgaggacccggctaggctggcggggttgccttactggttagcagaatgaatcaccgatacgcgagcgaacgtgaagcgactgctg
ctgcaaaacgtctgcgacctgagcaacaacatgaatggtcttcggtttccgtgtttcgtaaagtctggaaacgcggaagtcagcgccct
gcaccattatgttccggatctgcatcgcaggatgctgctggctaccctgtggaacacctacatctgtattaacgaagcgctggcattgac
cctgagtgatttttctctggtcccgccgcatccataccgccagttgtttaccctcacaacgttccagtaaccgggcatgttcatcatcagta
acccgtatcgtgagcatcctctctcgtttcatcggtatcattacccccatgaacagaaatcccccttacacggaggcatcagtgaccaaa
caggaaaaaaccgcccttaacatggcccgctttatcagaagccagacattaacgcttctggagaaactcaacgagctggacgcggat
gaacaggcagacatctgtgaatcgcttcacgaccacgctgatgagctttaccgcagctgcctcgcgcgtttcggtgatgacggtgaaa
acctctgacacatgcagctcccggagacggtcacagcttgtctgtaagcggatgccgggagcagacaagcccgtcagggcgcgtca
gcgggtgttggcgggtgtcggggcgcagccatgacccagtcacgtagcgatagcggagtgtatactggcttaactatgcggcatcag
agcagattgtactgagagtgcaccatatatgcggtgtgaaataccgcacagatgcgtaaggagaaaataccgcatcaggcgctcttcc
gcttcctcgctcactgactcgctgcgctcggtcgttcggctgcggcgagcggtatcagctcactcaaaggcggtaatacggttatccac
agaatcaggggafaacgcaggaaagaacatgtgagcaaaaggccagcaaaaggccaggaaccgtaaaaaggccgcgffgctggc
gtttttccataggctccgcccccctgacgagcatcacaaaaatcgacgctcaagtcagaggtggcgaaacccgacaggactataaag
ataccaggcgtttccccctggaagctccctcgtgcgctctcctgttccgaccctgccgcttaccggatacctgtccgcctttctcccttcg
ggaagcgtggcgctttctcatagctcacgctgtaggtatctcagttcggtgtaggtcgttcgctccaagctgggctgtgtgcacgaaccc
cccgttcagcccgaccgctgcgccttatccggtaactatcgtcttgagtccaacccggtaagacacgacttatcgccactggcagcag
ccactggtaacaggattagcagagcgaggtatgtaggcggtgctacagagttcttgaagtggtggcctaactacggctacactagaag
gacagtatttggtatctgcgctctgctgaagccagttaccttcggaaaaagagttggtagctcttgatccggcaaacaaaccaccgctgg
tagcggtggtttttttgtttgcaagcagcagattacgcgcagaaaaaaaggatctcaagaagatcctttgatcttttctacggggtctgacg
ctcagtggaacgaaaactcacgttaagggattttggtcatgaacaataaaactgtctgcttacataaacagtaatacaaggggtgttatga
gccatattcaacgggaaacgtcttgctctaggccgcgattaaattccaacatggatgctgatttatatgggtataaatgggctcgcgataa
tgtcgggcaatcaggtgcgacaatctatcgattgtatgggaagcccgatgcgccagagttgtttctgaaacatggcaaaggtagcgttg
ccaatgatgttacagatgagatggtcagactaaactggctgacggaatttatgcctcttccgaccatcaagcattttatccgtactcctgat
gatgcatggttactcaccactgcgatccccgggaaaacagcattccaggtattagaagaatatcctgattcaggtgaaaatattgttgatg
cgctggcagtgttcctgcgccggttgcattcgattcctgtttgtaattgtccttttaacagcgatcgcgtatttcgtctcgctcaggcgcaat
cacgaatgaataacggtttggttgatgcgagtgattttgatgacgagcgtaatggctggcctgttgaacaagtctggaaagaaatgcata
aacttttgccattctcaccggattcagtcgtcactcatggtgatttctcacttgataaccttatttttgacgaggggaaattaataggttgtatt
gatgttggacgagtcggaatcgcagaccgataccaggatcttgccatcctatggaactgcctcggtgagttttctccttcattacagaaa
cggctttttcaaaaatatggtattgataatcctgatatgaataaattgcagtttcatttgatgctcgatgagtttttctaagaattaattcatgag
cggatacatatttgaatgtatttagaaaaataaacaaataggggttccgcgcacatttccccgaaaagtgccacctgaaattgtaaacgtt
aatattttgttaaaattcgcgttaaatttttgttaaatcagctcattttttaaccaataggccgaaatcggcaaaatcccttataaatcaaaaga
atagaccgagatagggttgagtgttgttccagtttggaacaagagtccactattaaagaacgtggactccaacgtcaaagggcgaaaa
accgtctatcagggcgatggcccactacgtgaaccatcaccctaatcaagttttttggggtcgaggtgccgtaaagcactaaatcggaa
ccctaaagggagcccccgantagagcttgacggggaaagccggcgaacgtggcgagaaaggaagggaagaaagcgaaaggag
cgggcgctagggcgctggcaagtgtagcggtcacgctgcgcgtaaccaccacacccgccgcgcttaatgcgccgctacagggcgc
gtcccattcgcca
HA Tag

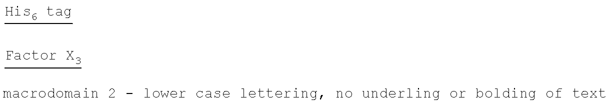

1. A method for detecting and/or quantifying mono- or poly-adenosine diphosphate ribose (ADP) transferase activity in an in vitro sample by measuring a detectable signal, the method comprising:
(1) contacting:
a) a recombinant macrodomain peptide comprising an epitope for antibody recognition,
b) an effective amount of a protein labeled with an ADP-ribose unit by ADP-ribosyltransferase,
c) an effective amount of adenosine diphosphate ribose (ADP)-ribosyltransferase,
d) an effective amount of nicotinamide adenine dinucleotide (NAD⁺), and
e) an effective amount of a detectably labeled antibody that binds the recombinant macrodomain peptide with the epitope for antibody recognition,
the contacting being under conditions that favor the formation of a complex comprising the detectably labeled antibody epitope bound to the recombinant macrodomain peptide that binds to a protein labeled with the ADP-ribose unit by ADP-ribosyltransferase;
(2) contacting the complex with an effective amount of a detectably labeled signal reagent; and
(3) measuring the detectable signal derived from the detectably labeled signal reagent, thereby detecting and/or quantifying mono- or poly-adenosine diphosphate ribose (ADP) transferase activity in the in vitro sample.

2. The method of claim 1, further comprising contacting a test agent with the recombinant macrodomain peptide with an epitope for antibody recognition, the ADP-ribosyltransferease, and NAD⁺.

3. The method of claim 1, wherein the epitopes are selected from the group of: HA tag, FLAG tag, His6 tag, myc-tag or a peptide or other agent that can be genetically fused with the macrodomain and recognized by their respective mono- and poly-clonal antibodies.

4. The method of claim 1, further comprising quenching the reaction prior to contacting the complex with the effective amount of the detectably labeled signal reagent.

5. The method of claim 2, wherein the test agent is a potential ADP-ribosyltransferase inhibitor.

6. The method of claim 1, wherein the ADP-ribosyltransferase enzyme is a PARP enzyme.

7. The method of claim 1, wherein the macrodomain is derived from a human macro-domain containing protein selected from the group of: MacroH2A1.1, MacroH2A1.2, MacroH2A2, PARP, ALC1, MacroD1, Macro D2, GDAP2, or C6orf130.

8. The method of claim 1, wherein the macrodomain is derived from a PARP selected from PARP14, PARP15, or PARP9.

9. The method of claim 1, wherein the macrodomain is macrodomain 1, macrodomain2, and macrodomain3 derived from PARP14.

10. The method of claim 1, further comprising repeating the method with varying amounts of ADP-ribosyltransferase enzyme.

11. The method of claim 1, wherein the signal emitted from the detectably labeled signal reagent is measured one or more times.

12. The method of claim 1, wherein the signal emitted from the detectably labeled signal reagent is measured multiple times and Km values are calculated using non-linear regression.

13. The method of claim 1, wherein the detectable signal of the detectably labeled signal reagent is from the group of: a colorimetric signal, a chemiluminescent signal, or a fluorescent signal.

14. A kit comprising the reagents for performing the methods of claim 1.

15. The kit of claim 14, further comprising instructions for carrying out the methods.


SEQUENCE LISTING (26 sequences)

SEQ ID NO: 1 - protein, 188 aa, Homo sapiens
KCFSRTVLAPGVVLIVQQGDLARLPVDVVVNASNEDLKHYGGLAAALSKAAGPELQADCDQIVKREGRLLPGNATISKAGKLPYHHVIHAVGPRWSGYEAPRCVYLLRRAVQLSLCLAEKYKYRSIAIPAISSGVFGFPLGRCVETIVSAIKENFQFKKDGHCLKEIYLVDVSEKTVEAFAEAVKTVF

SEQ ID NO: 2 - protein, 172 aa, Homo sapiens
DSGVYEMKIGSIIFQVASGDITKEEADVIVNSTSNSFNLKAGVSKAILECAGQNVERECSQQAQQRKNDYIITGGGFLRCKNIIHVIGGNDVKSSVSSVLQECEKKNYSSICLPAIGTGNAKQHPDKVAEAIIDAIEDFVQKGSAQSVKKVKVVIFLPQVLDVFYANMKKRE

SEQ ID NO: 3 - protein, 194 aa, Homo sapiens
SAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHAQSSSSSSRQLVP

SEQ ID NO: 4 - protein, 151 aa, Homo sapiens
ASSLNEDPEGSRITYVKGDLFACPKTDSLAHCISEDCRMGAGIAVLFKKKFGGVQELLNQQKKSGEVAVLKRDGRYIYYLITKKRASHKPTYENLQKSLEAMKSHCLKNGVTDLSMPRIGCGLDRLQWENVSAMIEEVFEATDIKITVYTL

SEQ ID NO: 5 - protein, 6 aa, Artificial Sequence
  Description of Artificial Sequence Synthetic 6xHis tag
HHHHHH

SEQ ID NO: 6 - DNA, 58 bp, Artificial Sequence
  Description of Artificial Sequence Synthetic primer
caccatcatcatcatcatattgagggtcgcgatatgaagcagcagaatttctgtgtgg

SEQ ID NO: 7 - DNA, 55 bp, Artificial Sequence
  Description of Artificial Sequence Synthetic primer
aagggcatcggtcgacttattattttctaaacgtaataaggtactctgggtatgc

SEQ ID NO: 8 - DNA, 55 bp, Artificial Sequence
  Description of Artificial Sequence Synthetic primer
tttctattgctacaaacgcatacgctatgcaccatcatcatcatcatattgaggg

SEQ ID NO: 9 - DNA, 49 bp, Artificial Sequence
  Description of Artificial Sequence Synthetic primer
ctcaagggcatcggtcgacttattaactaaccaaattgccgtttgcacg

SEQ ID NO: 10 - DNA, 72 bp, Artificial Sequence
  Description of Artificial Sequence Synthetic primer
tctagaaataattttgtttaactttaagaaggagatataccatgcaccatcatcatcatcatattgagggtc

SEQ ID NO: 11 - DNA, 40 bp, Artificial Sequence
  Description of Artificial Sequence Synthetic primer
ctcgagttattattttctaaacgtaataaggtactctggg

SEQ ID NO: 12 - DNA, 71 bp, Artificial Sequence
  Description of Artificial Sequence Synthetic primer
gtggtgctcgagttattaagcgtaatctggaacatcgtatgggtaactgacgagatttccattagcccttc

SEQ ID NO: 13 - DNA, 54 bp, Artificial Sequence
  Description of Artificial Sequence Synthetic primer
caccatcaccatcacattgaaggccgtaatcttcctgaacactggactgacatg

SEQ ID NO: 14 - DNA, 53 bp, Artificial Sequence
  Description of Artificial Sequence Synthetic primer
ctcaagggcatcggtcgacttattaagccgtgaaagttatgagatattctggg

SEQ ID NO: 15 - DNA, 69 bp, Artificial Sequence
  Description of Artificial Sequence Synthetic primer
tctagaaataattttgtttaactttaagaaggagatataccatgggccatcaccatcaccatcacattg

SEQ ID NO: 16 - DNA, 55 bp, Artificial Sequence
  Description of Artificial Sequence Synthetic primer
gtggtggtggtggtgctcgagttattaagccgtgaaagttatgagatattctggg

SEQ ID NO: 17 - DNA, 654 bp, Artificial Sequence
  Description of Artificial Sequence Synthetic polynucleotide
atgcaccatcatcatcatcatattgagggtcgcgggaaaacatcatgggaaaaaggaagcctggtgtccccgggaggcctgcagatgctgttggtgaaagagggtgtgcagaatgctaagaccgatgttgttgtcaactccgttcccttggatctcgtgcttagtagagggcctctttctaagtccctcttggaaaaagctggaccagagctccaggaggaattggacacagttggacaaggggtggctgtcagcatgggcacagtgctcaaaaccagcagctggaatctggactgtcgctatgtgcttcacgtggtagctccggagtggagaaatggtagcacatcttcactcaagataatggaagacataatcagagaatgtatggagatcactgagagcttgtccttaaaatcaattgcatttccagcaataggaacaggaaacttgggatttcctaaaaacatattcgctgaattaatcatttcagaggtgttcaaatttagtagcaagaatcagctgaaaactttacaagaggttcactttctgctgcacccgagtgatcatgaaaatattcaggcattttcagatgaatttgccagaagggctaatggaaatctcgtcagttacccatacgatgttccagattacgct

SEQ ID NO: 18 - protein, 218 aa, Artificial Sequence
  Description of Artificial Sequence Synthetic polypeptide
MHHHHHHIEGRGKTSWEKGSLVSPGGLQMLLVKEGVQNAKTDVVVNSVPLDLVLSRGPLSKSLLEKAGPELQEELDTVGQGVAVSMGTVLKTSSWNLDCRYVLHVVAPEWRNGSTSSLKIMEDIIRECMEITESLSLKSIAFPAIGTGNLGFPKNIFAELIISEVFKFSSKNQLKTLQEVHFLLHPSDHENIQAFSDEFARRANGNLVSYPYDVPDYA

SEQ ID NO: 19 - protein, 234 aa, Artificial Sequence
  Description of Artificial Sequence Synthetic polypeptide
MHHHHHHIEGRIQQQKTQDEMKENIIFLKCPVPPTQELLDQKKQFEKCGLQVLKVEKIDNEVLMAAFQRKKKMMEEKLHRQPVSHRLFQQVPYQFCNVVCRVGFQRMYSTPCDPKYGAGIYFTKNLKNLAEKAKKISAADKLIYVFEAEVLTGFFCQGHPLNIVPPPLSPGAIDGHDSVVDNVSSPETFVIFSGMQAIPQYLWTCTQEYVQSQDYSSGPMRPFAQHPWRGFASG

SEQ ID NO: 20 - DNA, 606 bp, Artificial Sequence
  Description of Artificial Sequence Synthetic polynucleotide
atgcaccatcatcatcatcatattgagggtcgcgatatgaagcagcagaatttctgtgtggtggagctgctgcctagtgatcctgagtacaacacggtggcaagcaagtttaatcagacctgctcacacttcagaatagagaagattgagaggatccagaatccagatctctggaatagctaccaggcaaagaaaaaaactatggatgccaagaatggccagacaatgaatgagaagcaactcttccatgggacagatgccggctccgtgccacacgtcaatcgaaatggctttaaccgcagctatgccggaaagaatgctgtggcatatggaaagggaacctattttgctgtcaatgccaattattctgccaatgatacgtactccagaccagatgcaaatgggagaaagcatgtgtattatgtgcgagtacttactggaatctatacacatggaaatcattcattaattgtgcctccttcaaagaaccctcaaaatcctactgacctgtatgacactgtcacagataatgtgcaccatccaagtttatttgtggcattttatgactaccaagcatacccagagtaccttattacgtttagaaaa

SEQ ID NO: 21 - protein, 202 aa, Artificial Sequence
  Description of Artificial Sequence Synthetic polypeptide
MHHHHHHIEGRDMKQQNFCVVELLPSDPEYNTVASKFNQTCSHFRIEKIERIQNPDLWNSYQAKKKTMDAKNGQTMNEKQLFHGTDAGSVPHVNRNGFNRSYAGKNAVAYGKGTYFAVNANYSANDTYSRPDANGRKHVYYVRVLTGIYTHGNHSLIVPPSKNPQNPTDLYDTVTDNVHHPSLFVAFYDYQAYPEYLITFRK

SEQ ID NO: 22 - DNA, 630 bp, Artificial Sequence
  Description of Artificial Sequence Synthetic polynucleotide
atgggccatcaccatcaccatcacattgaaggccgtaatcttcctgaacactggactgacatgaatcatcagctgttttgcatggtccagctagagccaggacaatcagaatataataccataaaggacaagttcacccgaacttgttcttcctacgcaatagagaagattgagaggatacagaatgcatttctctggcagagctaccaggtaaagaaaaggcaaatggatatcaagaatgaccataagaataatgagagactcctcttccatgggacagatgcagactcagtgccatatgtcaatcagcacggctttaatagaagttgtgctgggaaaaatgctgtatcctatggaaaaggaacctattttgctgtggatgccagttattctgccaaggacacctactccaagccagacagcaatgggagaaagcacatgtacgttgtgcgagtacttactggagtcttcacaaagggacgtgcaggattagtcacccctccacccaagaatcctcacaatcccacagatctctttgactcagtgacaaacaatacacgatctccaaagctatttgtggtattctttgataatcaggcttacccagaatatctcataactttcacggct

SEQ ID NO: 23 - protein, 210 aa, Artificial Sequence
  Description of Artificial Sequence Synthetic polypeptide
MGHHHHHHIEGRNLPEHWTDMNHQLFCMVQLEPGQSEYNTIKDKFTRTCSSYAIEKIERIQNAFLWQSYQVKKRQMDIKNDHKNNERLLFHGTDADSVPYVNQHGFNRSCAGKNAVSYGKGTYFAVDASYSAKDTYSKPDSNGRKHMYVVRVLTGVFTKGRAGLVTPPPKNPHNPTDLFDSVTNNTRSPKLFVVFFDNQAYPEYLITFTA

SEQ ID NO: 24 - DNA, 861 bp, Artificial Sequence
  Description of Artificial Sequence Synthetic polynucleotide
atgggccatcaccatcaccatcacattgaaggccgtggctgggcggccgccagggaggcggcgggccgcgacatgctggccgccgacctccggtgcagcctcttcgcctcggccctgcagagctacaagcgcgactcggtgctgcggcccttccccgcgtcctacgcccgcggcgactgtaaggactttgaagccctgcttgcagatgccagcaagttacctaacctgaaagaacttctccagtcctccggagacaaccacaaacgggcctgggacctggtgagctggattttatcctcaaaggtcctgacaatccacagtgcagggaaggcagagtttgaaaagatccaaaagctgactggggctcctcacacgcctgttcctgcaccggacttcctgtttgaaattgagtactttgacccagccaacgccaaattttatgagaccaaaggagaacgagacctaatctatgcatttcatggtagccgcctagaaaacttccattccattatccacaatggcctgcactgccatctgaacaagacatccttgttcggagaggggacctacctcaccagtgacttaagcctggccctcatatacagcccccatggccatgggtggcagcacagcctcctcggccccatccttagctgtgtggccgtgtgtgaggtcattgaccatccggacgtcaagtgccaaaccaagaagaaggattccaaggagatagatcgcagacgagcgagaatcaaacatagtgaagggggagacatccctcccaagtacttcgtggtcaccaataaccagctgctgcgagtgaagtacctcctggtgtattcacagaagccacccaagagggct

SEQ ID NO: 25 - protein, 287 aa, Artificial Sequence
  Description of Artificial Sequence Synthetic polypeptide
MGHHHHHHIEGRGWAAAREAAGRDMLAADLRCSLFASALQSYKRDSVLRPFPASYARGDCKDFEALLADASKLPNLKELLQSSGDNHKRAWDLVSWILSSKVLTIHSAGKAEFEKIQKLTGAPHTPVPAPDFLFEIEYFDPANAKFYETKGERDLIYAFHGSRLENFHSIIHNGLHCHLNKTSLFGEGTYLTSDLSLALIYSPHGHGWQHSLLGPILSCVAVCEVIDHPDVKCQTKKKDSKEIDRRRARIKHSEGGDIPPKYFVVTNNQLLRVKYLLVYSQKPPKRA

SEQ ID NO: 26 - DNA, 5893 bp, Artificial Sequence
  Description of Artificial Sequence Synthetic polynucleotide
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttattaagcgtaatctggaacatcgtatgggtaactgacgagatttccattagcccttctggcaaattcatctgaaaatgcctgaatattttcatgatcactcgggtgcagcagaaagtgaacctcttgtaaagttttcagctgattcttgctactaaatttgaacacctctgaaatgattaattcagcgaatatgtttttaggaaatcccaagtttcctgttcctattgctggaaatgcaattgattttaaggacaagctctcagtgatctccatacattctctgattatgtcttccattatcttgagtgaagatgtgctaccatttctccactccggagctaccacgtgaagcacatagcgacagtccagattccagctgctggttttgagcactgtgcccatgctgacagccaccccttgtccaactgtgtccaattcctcctggagctctggtccagctttttccaagagggacttagaaagaggccctctactaagcacgagatccaagggaacggagttgacaacaacatcggtcttagcattctgcacaccctctttcaccaacagcatctgcaggcctcccggggacaccaggcttcctttttcccatgatgttttcccgcgaccctcaatatgatgatgatgatggtgcatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca